(19) United States
(12) Patent Application Publication
(10) Pub. No.: US 2025/0225535 A1
(43) Pub. Date: Jul. 10, 2025

(54) METHOD FOR CALCULATING CARBON STORAGE IN MIXED FOREST ECOSYSTEM

(71) Applicants: NANJING FORESTRY UNIVERSITY, Nanjing (CN); INSTITUTE OF FOREST RESOURCE INFORMATION TECHNIQUES CAF, Beijing (CN)

(72) Inventors: Weifeng WANG, Nanjing (CN); Ke YAN, Nanjing (CN); Haikui LI, Beijing (CN); Honghua RUAN, Nanjing (CN); Menglin SU, Nanjing (CN); Xuehong MA, Nanjing (CN); Shuiqiang YU, Nanjing (CN); Tongli WANG, Nanjing (CN)

(73) Assignees: NANJING FORESTRY UNIVERSITY, Nanjing (CN); INSTITUTE OF FOREST RESOURCE INFORMATION TECHNIQUES CAF, Beijing (CN)

(21) Appl. No.: 19/023,641

(22) Filed: Jan. 16, 2025

Related U.S. Application Data
(63) Continuation of application No. PCT/CN2024/103935, filed on Jul. 5, 2024.

(30) Foreign Application Priority Data
Jan. 5, 2024 (CN) 202410019046.1

(51) Int. Cl. G06Q 30/018

(52) U.S. Cl. CPC G06Q 30/018

(57) ABSTRACT

A method for calculating carbon storage in a mixed forest ecosystem is provided. The method includes: acquiring basic geographic data, meteorological data, eco-physiological parameter, thinning management history data, and validation data; proposing an improved biome-biogeochemical cycles (Biome-BGC) model suitable for simulating carbon storage of a mixed forest ecosystem under management by improving a phenology module, adding a thinning operation management module, and optimizing the eco-physiological parameter, based on an existing Biome-BGC model; simulating, by taking a pine-oak mixed forest as a research object, the carbon storage based on the improved Biome-BGC model; validating the improved model; analyzing sensitivity of the eco-physiological parameter by an extended Fourier amplitude sensitivity test (EFAST) method; and selecting a highly sensitive parameter, and analyzing an effect of the highly sensitive parameter on the carbon storage by a path analysis method. The improved model exhibits good performance in calculating the carbon storage of the mixed forest.

### CROSS REFERENCE TO THE RELATED APPLICATIONS

This application is a continuation application of International Application No. PCT/CN2024/103935, filed on Jul. 5, 2024, which is based upon and claims priority to Chinese Patent Application No. 202410019046.1, filed on Jan. 5, 2024, the entire contents of which are incorporated herein by reference.

### TECHNICAL FIELD

The present disclosure belongs to the technical field of carbon sink estimation in a forest ecosystem, and specifically relates to a method for calculating carbon storage in a mixed forest ecosystem.

### BACKGROUND

As a major component of the terrestrial ecosystem, forests play an important role in balancing global carbon regulation and mitigating the rise in greenhouse gas (GHG) concentrations. Forest ecosystems have high carbon storage capacity, with annual fixed carbon storage accounting for over 60% of that in the entire terrestrial ecosystem. As a complex ecosystem composed of multiple tree species, mixed forests have great potential in carbon storage, and their carbon storage directly relates to global carbon balance and climate change. Appropriate forest management (such as thinning and fertilization) can optimize forest structure and increase productivity, thereby enhancing the carbon storage potential of mixed forests. Accurately simulating the carbon storage of mixed forests under management is a powerful guarantee for achieving sustainable forest development and carbon neutrality goals.

The biome-biogeochemical cycles (Biome-BGC) model is a widely used ecosystem process model that provides potential opportunities for estimating carbon cycling of the terrestrial ecosystem and its response to disturbances of long sequences and different scales. However, the Biome-BGC model can only simulate a single-plant functional type in a single grid, such as evergreen needle-leaved forest (ENF) or deciduous broad-leaved forest (DBF). For mixed forests, there are some shortcomings in setting existing phenology models as evergreen or deciduous models. A previous simulation study on mixed forests set phenology models as evergreen and deciduous separately and then weighted the average based on their relative ratio (Li and Sun, 2018). Although this method preserves the original structure of the model, it requires running the model twice, and more importantly, it leads to an inaccurate description of the carbon cycling process in the mixed forest, resulting in significant errors in carbon storage simulation. In addition, human management practices have a significant impact on the carbon cycling of forest ecosystems. Biome-BGC has expanded to cover the implementation of management and disturbance, such as mowing and grazing on grasslands, bamboo shoot harvesting in bamboo forests, and selective felling and fertilization (Mao et al., 2016). Previous studies related to thinning management have typically reduced the corresponding fixed values by defining a leaf area index (LAI) after thinning, thereby affecting the eco-physiological processes of vegetation (Hidy et al., 2012). Such a method only considers the changes in leaves after thinning and does not fully consider the biomass loss of various organs of vegetation. Therefore, Biome-BGC has limitations in accurately quantifying the carbon cycling process of mixed forests under thinning management. In order to effectively simulate the carbon storage of mixed forest ecosystems under management, there is an urgent need to improve the existing Biome-BGC model.

Eco-physiological parameters of different forest types are essential data for the operation of the Biome-BGC model. The parameterization process is a crucial step for applying the Biome-BGC model. However, at present, there are few studies on eco-physiological parameters, and most studies rely on literature data to determine these parameters (Liu et al., 2022b). Due to insufficient prior knowledge of specific eco-physiological parameters of mixed forests, the uncertainty of these parameters has had a negative impact on the accuracy of the model. Many studies have adopted automatic calibration models, such as simulated annealing (SA) (You et al., 2019), genetic algorithms (GAs) (Miyauchi et al., 2019), and model-independent parameter estimation and uncertainty analysis (PEST), for global parameter optimization. Therefore, it is an effective method to improve model accuracy by optimizing eco-physiological parameters through observation data optimization algorithms, thereby acquiring a set of parameters suitable for simulating carbon storage of mixed forests.

Identifying the most influential parameters and considering their interactions on carbon storage is crucial for the accurate calibration of the model (Ren et al., 2022). This will provide strong support for improving the application and development of the model. Sensitivity analysis is designed to quantify the extent to which changes in input parameters affect the output of the model. Some researchers have conducted global sensitivity analyses on carbon flux (Raj et al., 2014), carbon density of biomass pools (Miyauchi et al., 2019), and carbon storage (Robinson et al., 2009). Others have explored the sensitivity and uncertainty of parameters through machine learning (ML) methods (Dagon et al., 2020). These studies indicate that sensitive parameters vary with different species and regions. However, there is relatively little analysis on how changes in sensitive parameters of mixed forests affect simulation outputs. Therefore, identifying sensitive parameters that affect carbon storage and evaluating their impact on carbon storage is a key step in simulating the carbon cycling process of mixed forests.

Oaks (Quercus, with over 400 species) and pines (Pinus, with approximately 120 species) are two main forest-forming genera in temperate regions of the northern hemisphere. As a typical type of mixed forest, pine-oak mixed forests are widely distributed, mainly found from subtropical to temperate climate zones and in Mediterranean climate zones, with higher productivity, more stable ecosystems, and higher carbon sequestration potential. Therefore, based on the existing Biome-BGC model, the present disclosure proposes an improved Biome-BGC model for simulating carbon storage of mixed forest ecosystems under management. The present disclosure improves the phenology module, adds a thinning operation management module, and optimizes the eco-physiological parameters. The present disclosure simulates carbon storage in a pine-oak mixed forest through the improved model and validates the model. Meanwhile, the present disclosure identifies eco-physiological parameters with high sensitivity to carbon storage and evaluates their impact on carbon storage, which is of great significance for accurately simulating the carbon cycling of mixed forests.

### SUMMARY

In view of the above issues, a technical problem to be solved by the present disclosure is to provide a method for calculating carbon storage in a mixed forest ecosystem. The present disclosure improves the biome-biogeochemical cycles (Biome-BGC) model and optimizes eco-physiological parameters to enhance the applicability of the model to mixed forest ecosystems under management. The present disclosure explores the sensitive eco-physiological parameters that affect carbon storage in the mixed forest, and evaluates the impact of highly sensitive eco-physiological parameters on the carbon storage in the mixed forest.

In order to solve the above technical problem, the present disclosure adopts the following technical solution.

A method for calculating carbon storage in a mixed forest ecosystem includes the following steps:
S1: acquiring basic geographic data, meteorological data, eco-physiological parameter, thinning management history data, and validation data;
S2: proposing an improved Biome-BGC model suitable for simulating carbon storage of a mixed forest ecosystem under management by improving a phenology module, adding a thinning operation management module, and optimizing the eco-physiological parameter, based on an existing Biome-BGC model, specifically:
S201: developing a phenology model suitable for simulating the mixed forest by improving the phenology module based on an evergreen phenology model, specifically:
S2011: calculating start and end times of a transfer period of deciduous vegetation by defining a parameter, specifically a proportion of a transfer growth period to a growing season of the deciduous vegetation;
S2012: calculating a daily transfer amount of the mixed forest in different phenological periods by defining a parameter, specifically a ratio of evergreen vegetation to the deciduous vegetation;
S2013: describing start and end times of a litterfall process of the deciduous vegetation by defining a parameter, specifically a proportion of the litterfall process to the growing season of the deciduous vegetation; and calculating a daily litterfall amount of the mixed forest in different phenological periods based on the ratio parameter of the evergreen vegetation to the deciduous vegetation;
S202: adding the thinning operation management module to simulate an impact of thinning on the carbon storage of the mixed forest; and
S203: optimizing and analyzing the eco-physiological parameter through a flower pollination algorithm (FPA), and establishing a set of parameters suitable for simulating the carbon storage of the mixed forest;
S3: simulating, by taking the mixed forest as a research object, the carbon storage based on the improved Biome-BGC model;
S4: validating the improved Biome-BGC model;
S5: analyzing sensitivity of the eco-physiological parameter by an extended Fourier amplitude sensitivity test (EFAST) method; and
S6: selecting a highly sensitive parameter, and analyzing positive and negative effects of the highly sensitive parameter on the carbon storage by a path analysis method.

Preferably, the basic geographic data includes: digital elevation model (DEM), slope, aspect, soil sand content, clay content, silt content, shortwave albedo, nitrogen deposition, and nitrogen fixation;
the meteorological data includes: daily maximum temperature, daily minimum temperature, daily average temperature, daily precipitation, daily saturated vapor pressure deficit, daily shortwave radiation flux density, and day length;
regarding the eco-physiological parameter, three parameters, including the ratio of the evergreen vegetation to the deciduous vegetation, the proportion of the transfer growth period to the growing season of the deciduous vegetation, and the proportion of the litterfall process to the growing season of the deciduous vegetation, are added based on the eco-physiological parameter defined by the original model to calculate the transfer amount and the litterfall;
regarding the thinning management history data, 10 parameters are defined in the thinning operation management module, including thinning day, as well as thinning rates and transport rates of various plant organs; and
the validation data involves a validation based on measured data.

Preferably, specifically, in the step S2011: calculating start and end times of a transfer period of deciduous vegetation by defining a parameter, specifically a proportion of a transfer growth period to a growing season of the deciduous vegetation:
for the deciduous vegetation, the transfer period is described based on a start and end of the growing season; when a sum of daily average soil temperatures exceeds a defined critical value, a leaf starts to expand; and an actual leaf onset day is 15 days earlier than a calculated leaf onset day, marking the start of the growing season:

$${ST}_{soil} = {\sum }_{i = 1}^{m} {Tsoil\_avg}_{i} ( when Tsoil\_avg > 0 , m \leq 365 )$$

$${T}_{crit} = {e}^{4.795 + 0.129 * {T}_{avg}}$$

$${onset}_{day} = m ( {ST}_{soil} \geq {T}_{crit} )$$

$${Act}_{onset \_ day} = {onset}_{day} - 15$$

where, Tsoil_avgᵢ denotes an average soil temperature on an i-th day of a year; STₛₒᵢₗ denotes a sum of daily average soil temperatures when an average soil temperature is greater than 0; T_{avg} denotes an average of daily average temperatures within operating days; T_{crit} denotes the defined critical value; onset_{day} denotes the calculated leaf onset day; Act_{onset_day} denotes the actual leaf onset day; and m denotes a day when STₛₒᵢₗ is greater than or equal to T_{crit};
if, after July 1st, the day length is less than 10 hours and 55 minutes, and a soil temperature is lower than an average soil temperature in autumn or lower than 2° C., all leaves fall; and an actual leaf offset day is 15 days later than a calculated leaf offset day, marking the end of the growing season;
all the leaves fall when one of following conditions is met:

$$\{ \begin{matrix}{Daylen}_{j} \leq 39300 AND {Tsoil\_avg}_{j} \leq \\ {Tsoil}_{avg \_ aut} ( Sept . and Oct . ) ( j \geq 182 ) \\ {Tsoil\_avg}_{j} < 2 ( j \geq 182 )\end{matrix}$$

$${offset}_{day} = j$$

$${Act}_{offset \_ day} = {offset}_{day} + 15$$

where, Daylenⱼ denotes a day length of a j-th day of a year; Tsoil_avgⱼ denotes an average soil temperature on the j-th day of the year; Tsoil_{avg_aut} denotes an average soil temperature between September and October; offset_{day} denotes the calculated leaf offset day; Act_{offset_day} denotes the actual leaf offset day; and the growing season is calculated based on the actual leaf onset day and leaf offset day;

$$ngrowthdays = {Act}_{offset \_ day} - {Act}_{onset \_ day}$$

$${t}_{1} = {Act}_{onset \_ day}$$

$${t}_{2} = {Act}_{offset \_ day} + ngrowthdays \times {T}_{t \_ d}$$

where, ngrowthdays denotes a number of days in the growing season; t₁ and t₂ denote a start day and an end day of the transfer period of the deciduous vegetation, respectively; and T_{t_d} denotes the proportion of the transfer growth period to the growing season of the deciduous vegetation.

Preferably, specifically, in the step S2012: calculating a daily transfer amount of the mixed forest in different phenological periods by defining a parameter, specifically a ratio of evergreen vegetation to the deciduous vegetation:

$${S}_{daily \_ transfer} = \{ \begin{matrix}{C}_{transfer} / ndays\_E ( 1 \leq nday \leq {t}_{1} ) \\ \frac{E : D}{1 + E : D} \times {C}_{transfer} / ndays\_E + \frac{1}{1 + E : D} \times \\ 2 \times {C}_{transfer} / ndays\_D ( {t}_{1} \leq nday \leq {t}_{2} ) \\ \frac{E : D}{1 + E : D} \times {C}_{transfer} / ndays\_E ( {t}_{2} \leq nday \leq {Act}_{offset \_ day} ) \\ {C}_{transfer} / ndays\_E ( {Act}_{offset \_ day} \leq nday \leq 365 )\end{matrix}$$

where, S_{daily_transfer} denotes the daily transfer amount of the mixed forest; C_{transfer} denotes a transfer amount of each plant organ in the mixed forest; ndays_E denotes a number of remaining days for the transfer of the evergreen vegetation; ndays_D denotes a number of remaining days for the transfer of the deciduous vegetation; E:D denotes the ratio of the evergreen vegetation to the deciduous vegetation; and nday denotes a day of the year.

Preferably, specifically, in the step S2013: describing start and end times of a litterfall process of the deciduous vegetation by defining a parameter, specifically a proportion of the litterfall process to the growing season of the deciduous vegetation:

$${t}_{3} = {Act}_{offset \_ day} - ( ngrowthdays \times {LFG}_{d} ) + 1$$

$${t}_{4} = {Act}_{offset \_ day}$$

where, t₃ and t₄ denote a start day and an end day of the litterfall process of the deciduous vegetation, respectively; and LFG_{d} denotes the proportion of the litterfall process to the growing season of the deciduous vegetation;

specifically, in the step: calculating a daily litterfall amount of the mixed forest in different phenological periods based on the ratio parameter of the evergreen vegetation to the deciduous vegetation:

$${S}_{daily \_ litterfall} = \{ \begin{matrix}{C}_{litterfall \_ increment \_ E} ( 1 \leq nday \leq {Act}_{onset \_ day} ) \\ \frac{E : D}{1 + E : D} \times {C}_{litterfall \_ increment \_ E} ( {Act}_{onset \_ day} \leq nday \leq {t}_{3} ) \\ {C}_{litterfall \_ increment \_ E} \times \frac{E : D}{1 + E : D} + {G}_{litterfall \_ increment \_ D} \times \\ \frac{1}{1 + E : D} ( {t}_{3} \leq nday \leq {t}_{4} ) \\ {C}_{litterfall \_ increment \_ E} ( {t}_{4} \leq nday \leq 365 )\end{matrix}$$

$${C}_{litterfall \_ increment \_ E} = \frac{E : D}{1 + E : D} \times {C}_{annmax} \times F\_turnover / 365.$$

$$Drate = 2. \times ( {C}_{leaf \_ froot} - {C}_{litterfall \_ increment {\_ D}_{t}} \times {litdays}_{D} ) / {litdays}_{D}^{2}$$

$${C}_{litterfall \_ increment {\_ D}_{t + 1}} = {C}_{litterfall \_ increment {\_ D}_{t}} + Drate$$

where, S_{daily_litterfall} denotes the daily litterfall amount; C_{litterfall_increment_E} denotes a daily litterfall amount from the evergreen plant, remaining constant throughout the year; C_{litterfall_increment_D} denotes a daily litterfall amount from the deciduous vegetation; Cₐₙₙₘₐₓ denotes an annual maximum daily carbon content; F_turnover denotes an annual turnover rate; C_{leaf_froot} denotes a carbon content in a leaf or fine root; litdays_{D} denotes a number of remaining days for the deciduous vegetation to fall; Drate denotes a linear growth rate; t denotes a number of days required to remove all fine roots and leaves, as well as a number of iterations in an equation; C_{litterfall_increment_D}_{ₜ} denotes a daily litterfall amount from the deciduous vegetation on a t-th day; and C_{litterfall_increment_D}_{ₜ₊₁} denotes a daily litterfall amount from the deciduous vegetation on a (t+1)-th day.

Preferably, specifically, the step S202: adding the thinning management module to simulate an impact of thinning on the carbon storage of the mixed forest includes:
defining a thinning day, as well as thinning rate and transport rate parameters of various plant organs, where the thinning rate refers to a proportion of biomass removed during thinning, while the transport rate refers to a proportion of biomass removed from a site after thinning and no longer participating in carbon cycling after transportation is completed; and
calculating, based on the thinning rate and transport rate of each plant organ, a carbon loss and carbon content entering a next litterfall pool, specifically:

$${C}_{all \_ to \_ THN} = {C}_{all} \times THN$$

$${C}_{all \_ to \_ THN \_ litr} = {C}_{all \_ to \_ THN} \times ( 100 - TRAN ) / 100 \times litter$$

where, THN denotes the thinning rate; Cₐₗₗ denotes the carbon content of each organ; C_{all_to_THN} describes a carbon flux generated by thinning of each organ and is considered as the carbon loss of each organ; TRAN denotes the transport rate of each organ; litter denotes a ratio of each organ entering four litterfall pools; and C_{all_to_THN_litr} describes carbon that remains on site after thinning and is converted into a corresponding litterfall component.

Preferably, specifically, the step S203: optimizing and analyzing the eco-physiological parameter through a flower pollination algorithm (FPA), and establishing a set of parameters suitable for simulating the carbon storage of the mixed forest includes:
setting a maximum number of iterations, a population size, and a transfer probability; setting an objective function as a minimum residual sum between a simulated value and a measured value of the carbon storage, with a decision variable being the eco-physiological parameter to be optimized; and repeatedly adjusting a value of the parameter within a feasible range until the objective function reaches an ideal minimum value.

Preferably, the step S3: simulating, by taking the mixed forest as a research object, the carbon storage based on the improved Biome-BGC model includes:
first step: performing a spin-up process to make the model enter a stable state and output a restart file; and second step: inputting the restart file into the model, running the model forward from hence, and starting the thinning management module in a corresponding area where thinning operation management is implemented to simulate the impact of thinning on the carbon storage.

Preferably, the step S4: validating the improved Biome-BGC model includes:
first step: setting the phenology module of the original model as evergreen phenology and deciduous phenology respectively for simulation, performing weighted average according to the ratio of the mixed forest, and comparing a simulation result with a simulation result of an improved model formed by adjusting the phenology module; and
second step: comparing the simulation result of the improved model formed by adjusting the phenology module with a simulation result of an improved model formed by adjusting the phenology module and adding the thinning module, where all simulation processes are performed based on the optimized eco-physiological parameter; and
calculating R², mean absolute error (MAE), and root mean square error (RMSE) to evaluate the simulation result of the improved Biome-BGC model:

$${R}^{2} = {[ \frac{{\sum }_{i = 1}^{n} ( {simulated}_{i} - {simulated}_{min} ) ( {observed}_{i} - {observed}_{min} )}{\sqrt{{\sum }_{i = 1}^{n} {( {simulated}_{i} - {simulated}_{min} )}^{2} {( {observed}_{i} - {observed}_{min} )}^{2}}} ]}^{2}$$

$$MAE = \frac{1}{n} {\sum }_{i = 1}^{n} \vert {simulated}_{i} - {observed}_{i} \vert$$

$$RMSE = \sqrt{\frac{1}{n} {\sum }_{i = 0}^{n} {( {simulated}_{i} - {observed}_{i} )}^{2}}$$

where, n denotes a number of measured data; R² reflects a degree of fitting; MAE and RMSE reflect a degree of difference; R² closer to 1 leads to lower MAE and RMSE, indicating a higher simulation accuracy.

Preferably, in the step S5: analyzing sensitivity of the eco-physiological parameter by an EFAST method:

$$Y = f ( X ) = f ( {X}_{1} , {X}_{2} , {X}_{3} , … , {X}_{n} )$$

$${V}_{Y} = {\sum }_{i} {V}_{i} + {\sum }_{i} {\sum }_{j > i} {V}_{ij} + {\sum }_{i} {\sum }_{j > i} {\sum }_{k > j} {V}_{ijk} + … + {V}_{12 … n}$$

$${S}_{i} = {V}_{i} / {V}_{Y}$$

$${S}_{iT} = {S}_{i} + {S}_{ij} + {S}_{ijk} + … + {S}_{12 … n}$$

where, Y denotes an output result of the improved Biome-BGC model; Xᵢ denotes each eco-physiological parameter within a given distribution range; V_{Y} denotes a total variance of the output; Vᵢ denotes a variance of a single parameter; Vᵢⱼ−V_{12 . . . n} denotes a variance of an interaction between parameters; Sᵢ denotes a first-order sensitivity index, indicating a direct contribution of the parameter to the total variance of the output result; and S_{iT} denotes a global sensitivity index, representing a sum of first-order sensitivity of the parameter and sensitivity indices of each order for the interaction between the parameter and another parameter.

Beneficial Effects. Compared with the prior art, the present disclosure has the following advantages.
(1) The present disclosure introduces the growing season of the deciduous vegetation into the evergreen phenology module to calculate the transfer and litterfall processes of the mixed forest at different periods, thereby modifying the phenology module. The optimized phenology module helps to comprehensively capture the dynamic changes of the mixed forest ecosystem so as to improve the understanding of internal processes of the ecosystem, thereby enhancing the accuracy of the simulation result of the Biome-BGC model for ecological processes such as carbon cycling in the mixed forest.
(2) On the basis of improving the phenology module, the present disclosure incorporates the thinning operation management module into the model, which helps to simulate the impact of thinning on the carbon cycling of the mixed forest and changes in the carbon storage under different thinning management scenarios in the future. This helps to develop a sustainable forest management plan and balance economic and ecological benefits.
(3) The present disclosure establishes a set of parameters suitable for simulating the carbon storage of the mixed forest by optimizing and simulating the eco-physiological parameter of the mixed forest, which compensates for the value uncertainty of the eco-physiological parameter of the mixed forests and enhances the universality and practicality of the model. The optimized eco-physiological parameter helps deepen the understanding of the functions and structures of the mixed forest ecosystem, thereby improving the accuracy of the model in simulating ecological processes of the mixed forest.
(4) The present disclosure can understand which eco-physiological parameter is most sensitive to the impact on the simulation result, and can perform more accurate parameter calibration, thereby improving the accuracy of the model. The present disclosure helps to develop a more effective ecosystem management strategy to maximize the maintenance or enhancement of the carbon storage.
(5) The present disclosure can evaluate the positive and negative effects of the highly sensitive eco-physiological parameter on the carbon storage of the mixed forest, and can provide a deeper understanding of the contributions of various parameters to the carbon storage. Therefore, the present disclosure provides a theoretical basis for subsequent model parameter optimization and calibration, and further deepens the understanding of the internal processes and dynamics of the mixed forest ecosystem.

### BRIEF DESCRIPTION OF THE DRAWINGS

FIGS. 1A-1B are schematic diagrams showing a location of an experimental area; FIG. 1A shows detailed characteristics of the Qinling Mountains, FIG. 1B shows thinning sites and carbon storage validation sites in the Qinling Mountains (note: T+year represents thinning management performed in the specified year, for example, T2006 represents thinning performed in 2006);

FIG. 2 is a flowchart of a method for calculating carbon storage in a mixed forest ecosystem;

FIG. 3 is a flowchart of step S2 of the method for calculating carbon storage in a mixed forest ecosystem;

FIG. 4 is a flowchart of step S201 of the method for calculating carbon storage in a mixed forest ecosystem;

FIG. 5 is a conceptual diagram of simulating carbon cycling in a mixed forest through an improved Biome-BGC model;

FIGS. 6A-6C are validation diagrams of an improved model formed by modifying a phenology module;

FIGS. 7A-7B are validation diagrams of an improved model formed by modifying the phenology module and introducing a thinning module;

FIG. 8 is a schematic diagram of a first-order sensitivity index and a total sensitivity index of the carbon storage; and

FIG. 9 shows a path analysis model between a highly sensitive parameter and the carbon storage.

### DETAILED DESCRIPTION OF THE EMBODIMENTS

The present disclosure is further elucidated below in conjunction with specific embodiments, and embodiments are implemented under the premise of the technical solutions of the present disclosure. It should be understood that these embodiments are provided merely to illustrate the present disclosure rather than to limit the scope of the present disclosure.

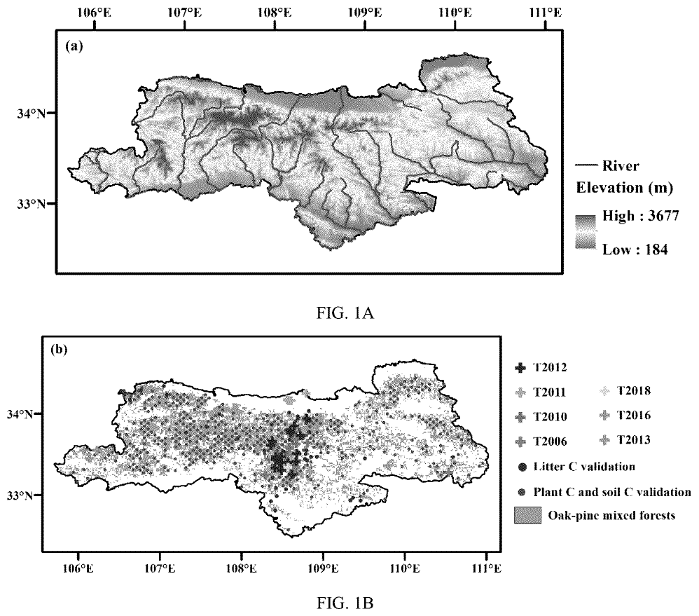

As shown in FIGS. 1A-1B, in the present disclosure, the Qinling Mountains are taken as the experimental area, and the pine-oak mixed forest is taken as the research object. The Qinling Mountains are mainly located in the southern part of Shaanxi Province, China (32°28′N to 34°40′N, 105°28′E to 113°3′E), with an area of approximately 63,700 km². As an important geographical boundary between the north and south of China, the Qinling Mountains are a transferal zone of vegetation between the north and south of China. The northern part of the Qinling Mountains is in a warm temperate semi-humid climate with an annual precipitation of 520 mm and an annual temperature of 10° C., while the southern part is in a subtropical humid climate with an annual precipitation of 820 mm and an annual temperature of 14° C. The Qinling Mountains have an altitude of less than 4,000 m, with undulating terrain, gradually rising from east to west. As the most extensive forest ecosystem in central China, the Qinling Mountains play a crucial role in forest and ecological conservation. Pinus tabulaeformis and quercus aliena var acutissima are the main species in the widely distributed mixed forests of pinus tabulaeformis and quercus aliena var acutissima in the Qinling Mountains, with a wide distribution range and playing an important role in the ecosystem. From 2000 to 2019, important conservation and management practices, namely low-intensity thinning management, were implemented to maintain ecological stability and restore zonal climax communities.

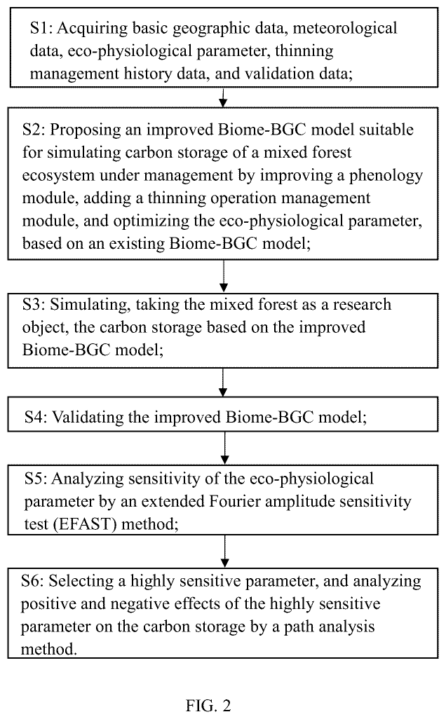

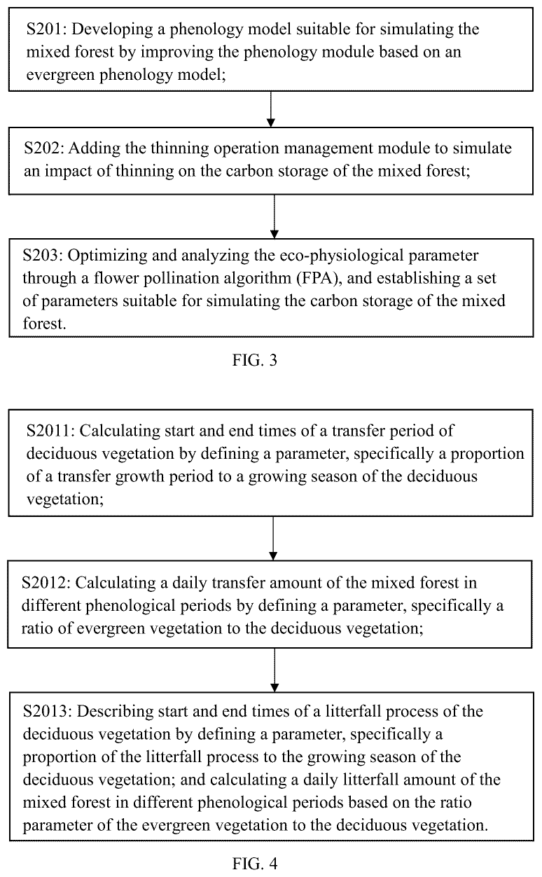

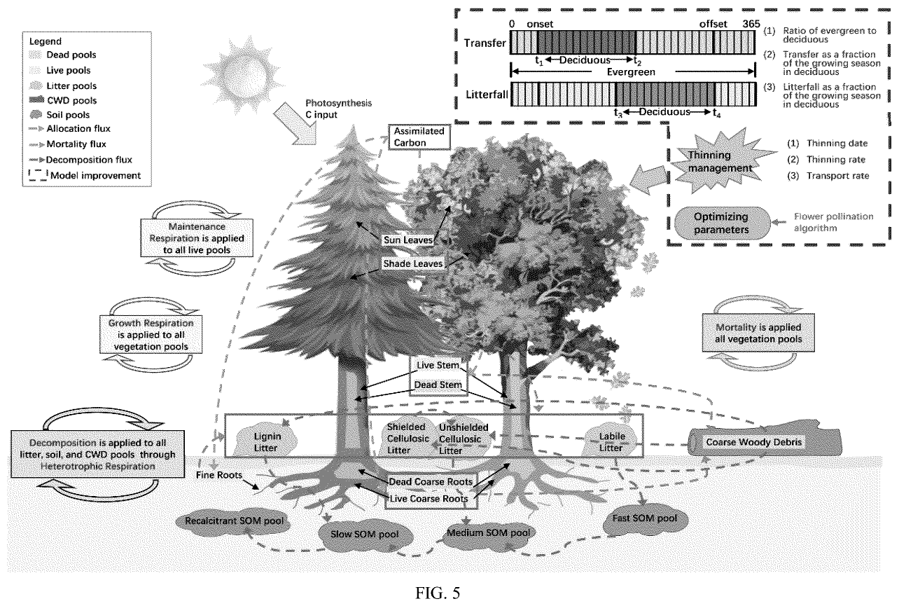

As shown in FIGS. 2 and 3, the present disclosure proposes a method for calculating carbon storage in a mixed forest ecosystem includes the following steps.
S1. Basic geographic data, meteorological data, eco-physiological parameter, thinning management history data, and validation data are acquired.
(1) The basic geographic data includes: digital elevation model (DEM), slope, aspect, soil sand content, clay content, silt content, shortwave albedo, nitrogen deposition, and nitrogen fixation. DEM is provided by the Resource and Environmental Science Data Center of the Chinese Academy of Sciences (https://www.resdc.cn/). The slope and aspect data are exported using ArcGIS based on DEM data, with a resolution of 1 km. As for the Chinese Soil Dataset (v1.1) from the Harmonized World Soil Database (HWSD) (2009) (http://westdc.westgis.ac.cn/zh-hans), the data source in China is the 1:1,000,000 soil data provided by the Institute of Soil Science, Chinese Academy of Sciences from the Second National Land Survey (FAO, 2019), with a resolution of 1 km. This dataset includes data on soil sand content, clay content, and silt content, as well as soil organic carbon content. The shortwave albedo comes from the measured data of a flux tower. Nitrogen deposition and nitrogen fixation are default data for the model.
(2) Regarding the meteorological data, the daily meteorological variables include seven indicators: daily maximum temperature, daily minimum temperature, daily average temperature, daily precipitation, daily saturated vapor pressure deficit, daily shortwave radiation flux density, and day length. The daily maximum temperature, daily minimum temperature, and daily precipitation from 1980 to 2019 are available from the China Meteorological Data Platform (http://data.cma.cn/) and interpolated using ANUSPLIN software. The Mountain Microclimate Simulation Model (MT-CLIM) is used to invert other indicators based on the above three variables. The concentration of atmospheric carbon dioxide comes from the observation dataset of the National Oceanic and Atmospheric Administration (NOAA) (http://www.noaa.gov/web.html) in the United States. All spatial data resolutions are resampled to 1 km, and the geographic coordinate systems are unified to WGS 1984 for consistent analysis.
(3) Regarding the eco-physiological parameter, the operation of the model requires 44 eco-physiological parameters from the original model. Three new parameters, namely the ratio of the evergreen vegetation to the deciduous vegetation, the proportion of the transfer growth period to the growing season of the deciduous vegetation, and the proportion of the litterfall process to the growing season of the deciduous vegetation, are defined in the improved model to calculate the transfer amount and the litterfall. Some parameters do not vary with the type of plant function, while the other 40 parameters need to be adjusted according to the pine-oak mixed forest. Most of the parameters are set based on previous research. However, due to the lack of data, it is necessary to optimize 17 parameters through an optimization algorithm so as to acquire a set of eco-physiological parameters suitable for the pine-oak mixed forest. Since the three parameters (the proportion of the transfer growth period to the growing season of the evergreen vegetation, the proportion of the litterfall process to the growing season of the evergreen vegetation, and the annual fire mortality rate) are fixed values, sensitivity analysis is not required. Table 1 shows the eco-physiological parameters of the pine-oak mixed forest ecosystem.

TABLE 1
Eco-physiological parameters of the pine-oak mixed forest ecosystem

| | | | | | | Is it |
|---|---|---|---|---|---|---|
| | | | | | | sensitivity |
| | | | | | Data | analysis |
| Parameter | Description | Unit | Range | Value | source | necessary? |
| E:D | Evergreen:deciduous | ratio | [0.25, 4] | 1.6 | Study | Yes |
| | | | | | site | |
| Tₜ_{_{—}}_{d} | Transfer growth period as fraction | prop. | [0, 1] | Optimized | | Yes |
| | of growing season in deciduous | | | | | |
| LFG_{d} | Litterfall as fraction of growing | prop. | [0, 1] | Optimized | | Yes |
| | season in deciduous | | | | | |
| Tₜ_{_{—}}ₑ | Transfer growth period as fraction | prop. | — | 1 | Biome- | No |
| | of growing season in evergreen | | | | BGC | |
| | | | | | V4.2 | |
| LFGₑ | Litterfall as fraction of growing | prop. | — | 1 | Biome- | No |
| | season in evergreen | | | | BGC | |
| | | | | | V4.2 | |
| LFRT | Annual leaf and fine root turnover | 1/year | [0, 1] | Optimized | | Yes |
| | fraction | | | | | |
| LWT | Annual live wood turnover | 1/year | — | 0.7 | Biome- | Yes |
| | fraction | | | | BGC | |
| | | | | | V4.2 | |
| WPM | Annual whole-plant mortality | 1/year | [0.0, 0.1] | Optimized | | Yes |
| | fraction | | | | | |
| FM | Annual fire mortality fraction | 1/year | | 0 | Study | No |
| | | | | | site | |
| FRC:L [symbol] | New fine root C:new leaf C | ratio | [0.5, 1.5] | Optimized | | Yes |
| SC:LC | New stem C: new leaf C | ratio | [1, 4] | Optimized | | Yes |
| LWC:T [symbol] | New live wood C:new total | ratio | — | 0.1 | Biome- | Yes |
| | wood C | | | | BGC | |
| | | | | | V4.2 | |
| CRC:S [symbol] | New coarse root C:new stem C | ratio | [0.184, 0.232] | Optimized | | Yes |
| CGP | Current growth proportion | prop. | [0, 1] | Optimized | | Yes |
| C:N_{leaf} | C:N of leaves | kgC/kgN | [18, 40] | Optimized | | Yes |
| C:N [symbol] | C:N of leaf litter | kgC/kgN | [41, 90] | Optimized | | Yes |
| C:N_{fr} | C:N of fine roots | kgC/kgN | — | 42.0 | White et | Yes |
| | | | | | al. | |
| | | | | | (2000) | |
| C:N_{lw} | C:N of live wood | kgC/kgN | — | 50.0 | White et | Yes |
| | | | | | al. | |
| | | | | | (2000) | |
| C:N_{dw} | C:N of dead wood | kgC/kgN | — | 442.0 | White et | Yes |
| | | | | | al. | |
| | | | | | (2000) | |
| L_{lab} | Leaf litter labile proportion | DIM | | 0.39 | Empirical | Yes |
| | | | | | parameter | |
| L_{cel} | Leaf litter cellulose proportion | DIM | — | 0.44 | Empirical | Yes |
| | | | | | parameter | |
| L_{lig} | Leaf litter lignin proportion | DIM | — | 0.17 | Empirical | Yes |
| | | | | | parameter | |
| FR_{lab} | Fine root labile proportion | DIM | | 0.3 | Empirical | Yes |
| | | | | | parameter | |
| FR_{cel} | Fine root cellulose proportion | DIM | — | 0.45 | Empirical | Yes |
| | | | | | parameter | |
| FR_{lig} | Fine root lignin proportion | DIM | — | 0.25 | Empirical | Yes |
| | | | | | parameter | |
| DW_{cel} | Dead wood cellulose proportion | DIM | | 0.76 | Empirical | Yes |
| | | | | | parameter | |
| DW_{lig} | Dead wood lignin proportion | DIM | — | 0.24 | Empirical | Yes |
| | | | | | parameter | |
| Wᵢₙₜ | Canopy water interception | 1/LAI/d | [0.0, 0.1] | Optimized | | Yes |
| | coefficient | | | | | |
| k | Canopy light extinction | DIM | — | 0.5 | Empirical | Yes |
| | coefficient | | | | parameter | |
| LAI_{all:p} [symbol] | All-sided to projected leaf area | DIM | [1.5, 4.0] | Optimized | | Yes |
| | ratio | | | | | |
| SLA | Canopy average specific leaf area | m²/kgC | [10, 60] | Optimized | | Yes |
| SLAₛₕₐ [symbol] | Ratio of shaded SLA:sunlit SLA | DIM | — | 2.0 | White et | Yes |
| | | | | | al. | |
| | | | | | (2000) | |
| FLNR | Fraction of leaf N in Rubisco | DIM | [0, 1] | Optimized | | Yes |
| Gₛₘₐₓ | Maximum stomatal conductance | m/s | — | 0.005 | Su et al. | Yes |
| | | | | | (2015) | |
| G_{cut} | Cuticular conductance | m/s | [0.0, 0.0001] | Optimized | | Yes |
| G_{bl} | Boundary layer conductance | m/s | — | 0.01 | White et | Yes |
| | | | | | al. | |
| | | | | | (2000) | |
| LWPᵢ | Leaf water potential:start of | MPa | [−0.78, −0.272] | Optimized | | Yes |
| | conductance reduction | | | | | |
| LWP_{f} | Leaf water potential:complete | MPa | — | −2.3 | White et | Yes |
| | conductance reduction | | | | al. | |
| | | | | | (2000) | |
| VPDᵢ | Vapor pressure deficit:start of | Pa | [488, 1320] | Optimized | | Yes |
| | conductance reduction | | | | | |
| VPD_{f} | Vapor pressure deficit:complete | Pa | — | 4100 | White et | Yes |
| | conductance reduction | | | | al. | |
| | | | | | (2000) | |

[symbol]

indicates data missing or illegible when filed

(4) Regarding thinning management history data, 10 parameters are newly defined in the thinning operation management module, mainly including thinning day, as well as thinning rates and transport rates of various plant organs. The relevant data comes from previous research and forest nurturing operation design data of forestry bureaus, as shown in Table 2.

TABLE 2
Thinning management history data

| | | | | | Thinning | | |
|---|---|---|---|---|---|---|---|
| | | | | | rates of | | |
| | | | | | leaves, live | | |
| | | | | | stems, dead | | |
| | | | | | stems, live | Transport | |
| | | | | | coarse roots, | rates of | |
| | | | | | dead coarse | leaves, live | |
| Serial | | Longitude | Latitude | Thinning | roots, and | stems, and | |
| number | Location | (°, E) | (°, N) | day | fine roots | dead stems | Source |
| 1 | Huoditang | 108.42 | 33.42 | 2010 Apr. 15 | 5% | 95% | Li et |
| | forest | | | | | | al., 2015 |
| 2 | district | 108.44 | 33.43 | 2010 Apr. 15 | 10% | 95% | Li et |
| | | | | | | | al., 2015 |
| 3 | | 108.46 | 33.44 | 2010 Apr. 15 | 15% | 95% | Li et |
| | | | | | | | al., 2015 |
| 4 | | 108.45 | 33.42 | 2010 Apr. 15 | 20% | 95% | Li et |
| | | | | | | | al., 2015 |
| 5 | | 108.42 | 33.48 | 2010 Jul. 1 | 5% | 95% | Chang |
| | | | | | | | et al., |
| | | | | | | | 2015; |
| | | | | | | | Wang and |
| | | | | | | | Yang, |
| | | | | | | | 2013 |
| 6 | | 108.49 | 33.45 | 2010 Jul. 1 | 15% | 95% | Chang |
| | | | | | | | et al., |
| | | | | | | | 2015; |
| | | | | | | | Wang and |
| | | | | | | | Yang, |
| | | | | | | | 2013 |
| 7 | | 108.48 | 33.46 | 2010 Jul. 1 | 25% | 95% | Chang |
| | | | | | | | et al., |
| | | | | | | | 2015; |
| | | | | | | | Wang and |
| | | | | | | | Yang, |
| | | | | | | | 2013 |
| 8 | | 108.42 | 33.44 | 2010 Jul. 1 | 20% | 95% | Chang |
| | | | | | | | et al., |
| | | | | | | | 2015; |
| | | | | | | | Wang and |
| | | | | | | | Yang, |
| | | | | | | | 2013 |
| 9 | | 108.42 | 33.44 | 2012 Jun. 1 | 5% | 95% | Zhao |
| | | | | | | | et al., 2015 |
| 10 | | 108.42 | 33.47 | 2012 Jun. 1 | 10% | 95% | Zhao |
| | | | | | | | et al., 2015 |
| 11 | | 108.49 | 33.47 | 2012 Jun. 1 | 15% | 95% | Zhao |
| | | | | | | | et al., 2015 |
| 12 | | 108.42 | 33.47 | 2012 Jun. 1 | 20% | 95% | Zhao |
| | | | | | | | et al., 2015 |
| 13 | | 108.60 | 33.43 | 2011 Mar. 1 | 5% | 95% | Sun, |
| | | | | | | | 2013 |
| 14 | | 108.39 | 33.47 | 2011 Mar. 1 | 10% | 95% | Sun, |
| | | | | | | | 2013 |
| 15 | | 108.40 | 33.32 | 2011 Mar. 1 | 15% | 95% | Sun, |
| | | | | | | | 2013 |
| 16 | | 108.42 | 33.43 | 2011 Mar. 1 | 20% | 95% | Sun, |
| | | | | | | | 2013 |
| 17 | Dangchuan | 106.14 | 33.45 | 2016 Jul. 1 | 20% | 90% | Zhang |
| | forest | | | | | | et al., |
| | farm | | | | | | 2018 |
| 18 | | 106.07 | 33.55 | 2016 Jul. 1 | 30% | 90% | Zhang |
| | | | | | | | et al., |
| | | | | | | | 2018 |
| 19 | | 106.23 | 33.55 | 2016 Jul. 1 | 40% | 90% | Zhang |
| | | | | | | | et al., |
| | | | | | | | 2018 |
| 20 | Xunyangba | 108.40 | 33.62 | 2010 Jun. 1 | 5% | 90% | Chang |
| | forest | | | | | | et al., |
| | farm | | | | | | 2015 |
| 21 | | 108.81 | 33.64 | 2010 Jun. 1 | 15% | 90% | Chang |
| | | | | | | | et al., |
| | | | | | | | 2015 |
| 22 | | 108.59 | 33.30 | 2010 Jun. 1 | 25% | 90% | Chang |
| | | | | | | | et al., |
| | | | | | | | 2015 |
| 23 | | 108.50 | 33.46 | 2011 Apr. 15 | 5% | 90% | Meng |
| | | | | | | | et al., 2016 |
| 24 | | 108.38 | 33.49 | 2011 Apr. 15 | 10% | 90% | Meng |
| | | | | | | | et al., 2016 |
| 25 | | 108.34 | 33.75 | 2011 Apr. 15 | 15% | 90% | Meng |
| | | | | | | | et al., 2016 |
| 26 | | 108.33 | 33.31 | 2011 Apr. 15 | 20% | 90% | Meng |
| | | | | | | | et al., 2016 |
| 27 | | 108.36 | 33.61 | 2012 Mar. 1 | 5% | 90% | Duan |
| | | | | | | | et al., 2020 |
| 28 | | 108.68 | 33.51 | 2012 Mar. 1 | 10% | 90% | Duan |
| | | | | | | | et al., 2020 |
| 29 | | 108.38 | 33.66 | 2012 Mar. 1 | 15% | 90% | Yin et |
| | | | | | | | al., 2019 |
| 30 | | 108.60 | 33.76 | 2012 Mar. 1 | 20% | 90% | Yin et |
| | | | | | | | al., 2019 |
| 31 | | 108.62 | 33.25 | 2012 Mar. 1 | 25% | 90% | Yin et |
| | | | | | | | al., 2019 |
| 32 | Xinkuang | 108.67 | 33.63 | 2010 Jul. 1 | 5% | 90% | Chang |
| | forest | | | | | | et al., |
| | farm | | | | | | 2015 |
| 33 | | 108.29 | 33.25 | 2010 Jul. 1 | 15% | 90% | Chang |
| | | | | | | | et al., |
| | | | | | | | 2015 |
| 34 | | 108.34 | 33.44 | 2010 Jul. 1 | 25% | 90% | Chang |
| | | | | | | | et al., |
| | | | | | | | 2015 |
| 35 | | 108.56 | 33.44 | 2010 Jul. 1 | 15% | 90% | Li et |
| | | | | | | | al., 2023 |
| 36 | | 108.57 | 33.34 | 2018 Aug. 1 | 15% | 90% | Li et |
| | | | | | | | al., 2023 |
| 37 | Shagou | 108.60 | 33.79 | 2012 Mar. 1 | 5% | 95% | Wang |
| | forest | | | | | | et al., 2020 |
| 38 | farm | 108.67 | 33.41 | 2012 Mar. 1 | 10% | 95% | Wang |
| | | | | | | | et al., 2020 |
| 39 | | 108.65 | 33.85 | 2012 Mar. 1 | 15% | 95% | Wang |
| | | | | | | | et al., 2020 |
| 40 | | 108.80 | 33.94 | 2012 Mar. 1 | 20% | 95% | Wang |
| | | | | | | | et al., 2020 |
| 41 | | 108.72 | 33.73 | 2012 Mar. 1 | 25% | 95% | Wang |
| | | | | | | | et al., 2020 |
| 42 | | 108.61 | 33.84 | 2011 Sep. 1 | 5% | 95% | Wu et |
| | | | | | | | al., 2016 |
| 43 | | 108.61 | 34.16 | 2011 Sep. 1 | 10% | 95% | Wu, |
| | | | | | | | 2015 |
| 44 | | 108.79 | 34.25 | 2011 Sep. 1 | 15% | 95% | Wu, |
| | | | | | | | 2015 |
| 45 | | 108.58 | 34.16 | 2011 Sep. 1 | 20% | 95% | Wu et |
| | | | | | | | al., 2016 |
| 46 | | 108.56 | 34.17 | 2011 Sep. 1 | 25% | 95% | Wu et |
| | | | | | | | al., 2016 |
| 47 | Shangluo | 109.80 | 33.36 | 2013 Aug. 1 | 10% | 95% | Yu |
| | area | | | | | | and Zhang, |
| | | | | | | | 2016 |
| 48 | | 108.84 | 33.29 | 2013 Aug. 1 | 20% | 95% | Yu |
| | | | | | | | and Zhang, |
| | | | | | | | 2016 |
| 49 | | 110.37 | 33.72 | 2013 Aug. 1 | 30% | 95% | Yu |
| | | | | | | | and Zhang, |
| | | | | | | | 2016 |
| 50 | | 108.66 | 33.34 | 2006 May 1 | 10% | 95% | Ran |
| | | | | | | | et al., 2013 |
| 51 | | 110.54 | 33.28 | 2006 May 1 | 20% | 95% | Ran, |
| | | | | | | | 2013 |
| 52 | | 108.67 | 33.69 | 2006 May 1 | 30% | 95% | Yu et |
| | | | | | | | al., 2014 |
| 53 | National | 108.57 | 33.40 | 2012 Jul. 1 | 25% | 95% | Dou |
| | Field | | | | | | et al., 2015 |
| 54 | Science | 108.48 | 33.40 | 2012 Jul. 1 | 22% | 95% | Dou |
| | Research | | | | | | et al., 2015 |
| 55 | Station | 108.43 | 33.44 | 2012 Jul. 1 | 15% | 95% | Dou |
| | | | | | | | et al., 2015 |
| 56 | | 108.49 | 33.30 | 2012 Jul. 1 | 8% | 95% | Dou |
| | | | | | | | et al., 2015 |
| 57 | | 108.41 | 33.33 | 2012 Jul. 1 | 5% | 95% | Dou |
| | | | | | | | et al., 2015 |
| 58 | Pingheliang | 108.57 | 33.49 | 2010 Sep. 1 | 5% | 95% | Wu et |
| | Nature | | | | | | al., 2017 |
| 59 | Reserve | 108.59 | 33.58 | 2010 Sep. 1 | 15% | 95% | Wu et |
| | | | | | | | al., 2017 |
| 60 | | 108.39 | 33.50 | 2010 Sep. 1 | 25% | 95% | Wu et |
| | | | | | | | al., 2017 |
| 61 | National | 108.45 | 33.43 | 2012 Aug. 20 | 20% | 95% | Luo |
| | Field Science | | | | | | et al., 2017 |
| | Research | | | | | | |
| | Station | | | | | | |

(5) Regarding the validation data, for the plant carbon storage, the eco-physiological parameter is optimized based on 485 sites of measured data from the 6th (2003), 7th (2008), and 8th (2013) forest resource inventories of the Qinling Mountains. Model validation is performed based on the data from the ninth (2018) forest resource inventory. For the soil carbon storage, the measured data comes from the Soil Sub-center of the National Earth System Science Data Center (http://soil.geodata.cn) (Liu Feng, 2020). For the litterfall carbon storage, some measured data are shown in Table 3.

TABLE 3
Validation data of litterfall carbon storage

| Serial | Longitude | Latitude | | Litterfall carbon | |
|---|---|---|---|---|---|
| number | (°, E) | (°, N) | Year | storage (t · ha⁻¹) | Source |
| 1 | 107.95 | 33.69 | 2019 | 79.3275 | Zhao et al., 2022 |
| 2 | 107.09 | 34.23 | 2016 | 49.3824-50.968 | Yue and Yang, 2019 |
| 3 | 108.59 | 34.10 | 2011 | 1.01 | Wu et al., 2016 |
| 4 | 106.53 | 34.28 | 2017 | 38.6 | Wang et al., 2019 |
| 5 | 107.57 | 33.84 | 2012 | 2.52 | Shen et al., 2015 |
| 6 | 108.27 | 33.55 | 2012 | 3.28 | Shen et al., 2015 |
| 7 | 108.42 | 33.42 | 2012 | 2.41 | Shen et al., 2015 |
| 8 | 108.64 | 33.43 | 2012 | 2.59 | Shen et al., 2015 |
| 9 | 110.59 | 33.47 | 2012 | 1.03 | Shen et al., 2015 |
| 10 | 108.42 | 33.09 | 2010 | 11.265 | He et al., 2012 |
| 11 | 108.38 | 33.21 | 2010 | 3.047 | He et al., 2012 |
| 12 | 106.63 | 34.17 | 2015 | 9.6144-50.7136 | Li et al., 2017 |
| 13 | 106.57 | 34.24 | 2015 | 41.6144-50.7136 | Li et al., 2017 |
| 14 | 108.45 | 33.43 | 2012 | 9.33 | Li, 2014 |
| 15 | 108.42 | 32.81 | 2012 | 9.33 | Li et al., 2014 |
| 16 | 108.55 | 33.07 | 2014 | 12.139 | Kang et al., 2006 |
| 17 | 108.36 | 32.96 | 2005 | 254.8 | He et al., 2010 |
| 18 | 108.43 | 32.99 | 2005 | 256.61 | He et al., 2010 |
| 19 | 107.73 | 33.99 | 2009 | 2.7 | Guo et al., 2010 |
| 20 | 108.93 | 33.46 | 2010 | 1.75 | Wu, 2015 |
| 21 | 108.80 | 33.42 | 2010 | 1.68 | Wu, 2015 |
| 22 | 108.81 | 33.50 | 2010 | 1.64 | Wu, 2015 |
| 23 | 108.87 | 33.46 | 2010 | 1.59 | Wu, 2015 |
| 24 | 108.78 | 33.45 | 2010 | 1.91 | Wu, 2015 |
| 25 | 108.70 | 33.53 | 2010 | 1.77 | Wu, 2015 |
| 26 | 108.52 | 33.53 | 2010 | 1.7 | Wu, 2015 |
| 27 | 108.79 | 33.54 | 2010 | 1.64 | Wu, 2015 |
| 28 | 108.68 | 33.97 | 2011 | 1.26 | Wu, 2015 |
| 29 | 108.82 | 34.04 | 2011 | 1.2 | Wu, 2015 |
| 30 | 108.69 | 33.94 | 2011 | 1.2 | Wu, 2015 |
| 31 | 108.84 | 33.91 | 2011 | 1.09 | Wu, 2015 |
| 32 | 108.83 | 33.77 | 2011 | 0.84 | Wu, 2015 |
| 33 | 108.76 | 33.73 | 2011 | 0.87 | Wu, 2015 |
| 34 | 108.57 | 33.80 | 2011 | 0.83 | Wu, 2015 |
| 35 | 108.60 | 33.84 | 2011 | 0.79 | Wu, 2015 |
| 36 | 109.34 | 32.94 | 2017 | 76.7904 | Dong et al., 2020 |
| 37 | 109.36 | 32.97 | 2017 | 85.68 | Dong et al., 2020 |
| 38 | 109.44 | 32.88 | 2017 | 8.10784 | Dong et al., 2020 |
| 39 | 106.77 | 34.29 | 2015 | 49.4384 | Dong et al., 2017 |
| 40 | 107.06 | 34.34 | 2016 | 11.49-16.63 | Yue and Yang, 2019 |
| 41 | 108.49 | 33.38 | 2010 | 11.26558 | He, 2011 |
| 42 | 108.59 | 33.68 | 2010 | 3.047313 | He, 2011 |
| 43 | 108.49 | 33.38 | 2005 | 6.761 | Kang et al., 2006 |
| 44 | 108.37 | 33.87 | 2000 | 3.634 | Liu et al., 2002 |
| 45 | 107.32 | 33.77 | 2000 | 5.145 | Liu et al., 2002 |
| 46 | 109.56 | 33.43 | 2000 | 3.758 | Liu et al., 2002 |

S2. An improved Biome-BGC model suitable for simulating carbon storage of a mixed forest ecosystem under management is proposed by improving a phenology module, adding a thinning operation management module, and optimizing the eco-physiological parameter, based on a version 4.2 Biome-BGC model.

To improve the applicability of Biome-BGC in the mixed forest, the present disclosure assumes that the phenology model is an evergreen model before simulation and introduces the growing season of the deciduous forest into the evergreen phenology module so as to develop a phenology module suitable for the mixed forest.

The mixed forest ecosystem is disrupted by thinning management. Therefore, the present disclosure introduces the thinning operation management module into the model to simulate the impact of thinning on the carbon storage of the mixed forest.

Considering the uncertainty of specific values of the eco-physiological parameters in the mixed forest, the present disclosure combines measured data with the optimization algorithm to optimize the parameters. Through optimization, the present disclosure establishes a set of optimized eco-physiological parameters suitable for simulating the carbon storage of the mixed forest.
S201. Specifically, the phenology module is improved based on an evergreen phenology model as follows.
S2011. Start and end times of a transfer period of deciduous vegetation are calculated by defining a parameter, specifically a proportion of a transfer growth period to a growing season of the deciduous vegetation.

The daily phenology of the existing Biome-BGC model transfers carbon to new tissues during the growth period. The transfer period of the evergreen vegetation in the model is throughout the year. The present disclosure incorporates the transfer period of the deciduous vegetation into the evergreen model and updates the daily transfer amount during different phenological periods based on the ratio of the evergreen vegetation to the deciduous vegetation in the mixed forest.

For the deciduous vegetation, the transfer period is described based on a start and end of the growing season. When a sum of daily average soil temperatures (average soil temperature exceeds 0° C.) exceeds a defined critical value (STₛₒᵢₗ>T_{crit}), a leaf begins to expand. An actual leaf onset day is 15 days earlier than a calculated leaf onset day, marking the start of the growing season:

$${ST}_{soil} = {\sum }_{i = 1}^{m} {Tsoil\_avg}_{i} ( when Tsoil\_avg > 0 , m \leq 365 )$$

$${T}_{crit} = {e}^{4.795 + 0.129 * {T}_{avg}}$$

$${onset}_{day} = m ( {ST}_{soil} \geq {T}_{crit} )$$

$${Act}_{onset \_ day} = {onset}_{day} - 15$$

where, Tsoil_avgᵢ denotes an average soil temperature on an i-th day of a year; STₛₒᵢₗ denotes a sum of daily average soil temperatures when an average soil temperature is greater than 0; T_{avg} denotes an average of daily average temperatures within operating days; T_{crit} denotes the defined critical value; onset_{day} denotes the calculated leaf onset day; Act_{onset_day} denotes the actual leaf onset day; and m denotes a day when STₛₒᵢₗ is greater than or equal to T_{crit}.

If, after July 1st, the day length is less than 10 hours and 55 minutes (39,300 seconds), and a soil temperature is lower than an average soil temperature in autumn (September and October) or lower than 2° C., all leaves fall. An actual leaf offset day is 15 days later than a calculated leaf offset day, marking the end of the growing season.

All the leaves fall when one of following conditions is met:

$$\{ \begin{matrix}{Daylen}_{j} \leq 39300 AND {Tsoil\_avg}_{j} \leq \\ {Tsoil}_{avg \_ aut} ( Sept . and Oct . ) ( j \geq 182 ) \\ {Tsoil\_avg}_{j} < 2 ( j \geq 182 )\end{matrix}$$

$${offset}_{day} = j$$

$${Act}_{offset \_ day} = {offset}_{day} + 15$$

where, Daylenⱼ denotes a day length of a j-th day of a year; Tsoil_avgⱼ denotes an average soil temperature on the j-th day of the year; Tsoil_{avg_aut} denotes an average soil temperature between September and October; offset_{day} denotes the calculated leaf offset day; Act_{offset_day} denotes the actual leaf offset day; and the growing season is calculated based on the actual leaf onset day and leaf offset day.

In this embodiment, start and end times of a transfer period of deciduous vegetation are calculated by defining a new parameter, namely a proportion of a transfer growth period to a growing season of the deciduous vegetation (T_{t_d}, shown in Table 1):

$$ngrowthdays = {Act}_{offset \_ day} - {Act}_{onset \_ day}$$

$${t}_{1} = {Act}_{onset \_ day}$$

$${t}_{2} = {Act}_{offset \_ day} + ngrowthdays \times {T}_{t \_ d}$$

where, ngrowthdays denotes a number of days in the growing season; and t₁ and t₂ denote a start day and an end day of the transfer period of the deciduous vegetation, respectively.
S2012. A daily transfer amount of the mixed forest in different phenological periods is calculated by defining a parameter, specifically a ratio of evergreen vegetation to the deciduous vegetation.

In the present disclosure, a daily transfer amount of the mixed forest in different phenological periods is calculated by defining a ratio of evergreen vegetation to the deciduous vegetation (E:D, shown in Table 1). The calculation equation is as follows:

$${S}_{daily \_ transfer} = \{ \begin{matrix}{C}_{transfer} / ndays\_E ( 1 \leq nday \leq {t}_{1} ) \\ \frac{E : D}{1 + E : D} \times {C}_{transfer} / ndays\_E + \frac{1}{1 + E : D} \times \\ 2 \times {C}_{transfer} / ndays\_D ( {t}_{1} \leq nday \leq {t}_{2} ) \\ \frac{E : D}{1 + E : D} \times {C}_{transfer} / ndays\_E ( {t}_{2} \leq nday \leq {Act}_{offset \_ day} ) \\ {C}_{transfer} / ndays\_E ( {Act}_{offset \_ day} \leq nday \leq 365 )\end{matrix}$$

where, S_{daily_transfer} denotes the daily transfer amount of the mixed forest; C_{transfer} denotes a transfer amount of each plant organ (including leaves, fine roots, live stems, dead stems, live coarse roots, and dead coarse roots) in the mixed forest; ndays_E denotes a number of remaining days for the transfer of the evergreen vegetation; ndays_D denotes a number of remaining days for the transfer of the deciduous vegetation; and nday denotes a day of the year.
S2013. Start and end times of a litterfall process of the deciduous vegetation are described by defining a parameter, specifically a proportion of the litterfall process to the growing season of the deciduous vegetation, and a daily litterfall amount of the mixed forest in different phenological periods is acquired.

During the litterfall process, carbon is transferred from fine roots and leaves to four litterfall pools according to the ratios specified in Table 1. The evergreen vegetation produces litterfall every day of the year, and the litterfall process of the deciduous vegetation has significant seasonal variations. Therefore, the present disclosure defines the litterfall cycle of the deciduous vegetation in the model and calculates the daily litterfall amount based on the ratio of the evergreen vegetation to the deciduous vegetation in the mixed forest.

In the present disclosure, the start and end times of the litterfall process are described by defining a new parameter, namely a proportion of the litterfall process to the growing season of the deciduous vegetation (LFG_{d}, shown in Table 1).

$${t}_{3} = {Act}_{offset \_ day} - ( ngrowthdays \times {LFG}_{d} ) + 1$$

$${t}_{4} = {Act}_{offset \_ day}$$

where, t₃ and t₄ denote a start day and an end day of the litterfall process of the deciduous vegetation, respectively.

The daily litterfall amount of the mixed forest in different phenological periods is calculated as follows:

$${S}_{daily \_ litterfall} = \{ \begin{matrix}{C}_{litterfall \_ increment \_ E} ( 1 \leq nday \leq {Act}_{onset \_ day} ) \\ \frac{E : D}{1 + E : D} \times {C}_{litterfall \_ increment \_ E} ( {Act}_{onset \_ day} \leq nday \leq {t}_{3} ) \\ {C}_{litterfall \_ increment \_ E} \times \frac{E : D}{1 + E : D} + {G}_{litterfall \_ increment \_ D} \times \\ \frac{1}{1 + E : D} ( {t}_{3} \leq nday \leq {t}_{4} ) \\ {C}_{litterfall \_ increment \_ E} ( {t}_{4} \leq nday \leq 365 )\end{matrix}$$

$${C}_{litterfall \_ increment \_ E} = \frac{E : D}{1 + E : D} \times {C}_{annmax} \times F\_turnover / 365.$$

$$Drate = 2. \times ( {C}_{leaf\_froot} - {C}_{litterfall \_ increment {\_ D}_{t}} \times {litdays}_{D} ) / {litdays}_{D}^{2}$$

$${C}_{litterfall \_ increment {\_ D}_{t + 1}} = {C}_{litterfall \_ increment {\_ D}_{t}} + Drate$$

where, S_{daily-litterfall} denotes the daily litterfall amount; C_{litterfall_increment_E} denotes a daily litterfall amount from the evergreen plant (including leaves and fine roots), remaining constant throughout the year; C_{litterfall_increment_D} denotes a daily litterfall amount from the deciduous vegetation, increasing at a linear growth rate (Drate) from 0 such that all fine roots and leaves fall before t₄; Cₐₙₙₘₐₓ denotes an annual maximum daily carbon content; F_turnover denotes an annual turnover rate; C_{leaf_froot} denotes a carbon content in a leaf or fine root; litdays_{D} denotes a number of remaining days for the deciduous vegetation to fall; t denotes a number of days required to remove all fine roots and leaves, as well as a number of iterations in an equation; C_{litterfall_increment_D}_{ₜ} denotes a daily litterfall amount from the deciduous vegetation on a t-th day; and C_{litterfall_increment_D}_{ₜ₊₁} denotes a daily litterfall amount from the deciduous vegetation on a (t+1)-th day.
S202. The thinning operation management module is added to simulate an impact of thinning on the carbon storage of the mixed forest.

The present disclosure defines the following parameters. (1) thinning day (e.g. Apr. 20, 2001). (2) Thinning rate of various plant organs. It refers to the ratio of biomass removed during the thinning process, and is calculated as a percentage. There are mainly thinning rates of leaves, live stems, dead stems, live coarse roots, dead coarse roots, and fine roots. (3) Transport rate. It refers to the proportion of organs removed from the site after thinning and no longer participating in the carbon cycling after transportation is completed, and is calculated as a percentage. The parameter includes transport rates of leaves, live stems, and dead stems. In addition, the present disclosure defines that live coarse roots, dead coarse roots, and fine roots are not transported, but remain on site and become part of the dead wood or litterfall pool according to the root system type.

Based on the thinning rate and transport rate of each plant organ, a carbon loss and carbon content entering a next litterfall pool are calculated, specifically:

$${C}_{all \_ to \_ THN} = {C}_{all} \times THN$$

$${C}_{all \_ to \_ THN \_ litr} = {C}_{all \_ to \_ THN} \times ( 100 - TRAN ) / 100 \times litter$$

where, THN denotes the thinning rate; Cₐₗₗ denotes the carbon content of each organ; C_{all_to_THN} describes a carbon flux generated by thinning of each organ and is considered as the carbon loss of each organ; TRAN denotes the transport rate of each organ; litter denotes a ratio of each organ entering four litterfall pools, and is specified in Table 1; and C_{all_to_THN_litr} describes organs that remain on site after thinning and is converted into corresponding litterfall components. After the above functions are completed, the actual vegetation carbon pool and litterfall carbon pool are modified and updated.
S203. The eco-physiological parameter is optimized and analyzed through a flower pollination algorithm (FPA), and a set of parameters suitable for simulating the carbon storage of the mixed forest is established.

Taking the pine-oak mixed forest as the experimental object, the 17 eco-physiological parameters in Table 1 are optimized and analyzed based on a flower pollination algorithm (FPA). This algorithm is a popular intelligent optimization algorithm with the advantages of fast speed and being less prone to getting stuck in local extremes. The present disclosure sets the following parameters: maximum number of iterations N=50, number of individuals in the population n=20, and transfer probability p=0.8. The objective function is a minimum residual sum between the simulated value and the measured value of the carbon storage, with the decision variable being the 17 eco-physiological parameters. The values of the parameters are repeatedly adjusted within a feasible range until the objective function reaches an ideal minimum value, thereby completing the optimization of the eco-physiological parameters.
S3. By taking the pine-oak mixed forest as a research object, the carbon storage is simulated based on the improved Biome-BGC model.

The operation of the improved Biome-BGC model is divided into two steps. The first step is a spin-up process, which is performed to bring the model into a stable state and output a restart file. It typically requires a steady-state initial condition to ensure a balance between input and output fluxes and a balance between the system and the environment. In the second step, the model uses the restart file as input and runs forward from then on. The thinning management module is started in the corresponding area where the thinning measure is taken to simulate the impact of thinning on the carbon storage.

The Biome-BGC model is a one-dimensional model that simulates in point form. The present disclosure performs grid-by-grid simulation through code compilation to achieve region simulation. The carbon pool set by the present disclosure includes plant carbon storage, litterfall carbon storage, soil carbon storage, and total carbon storage.
S4. The improved Biome-BGC model is validated.

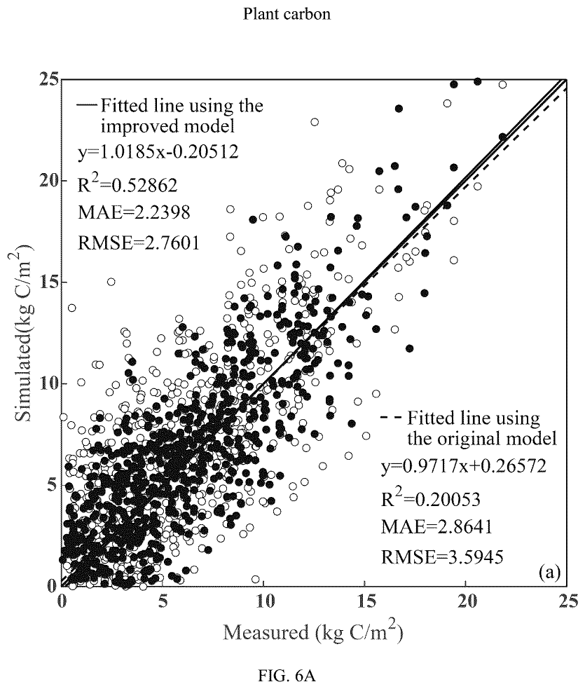

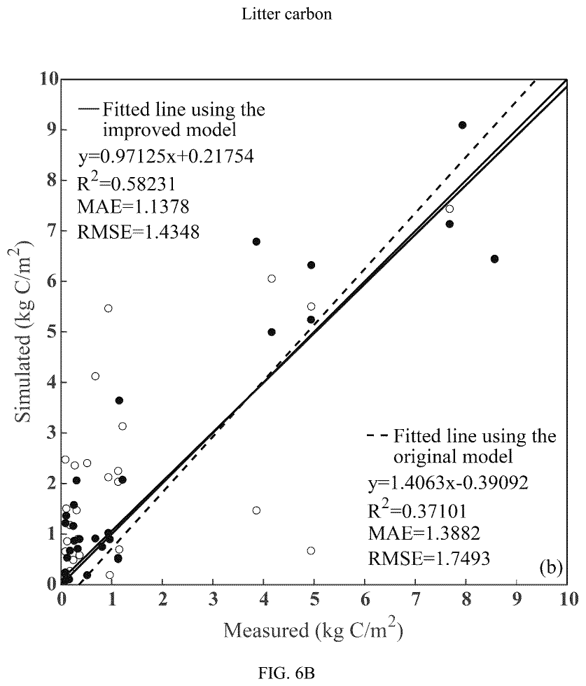

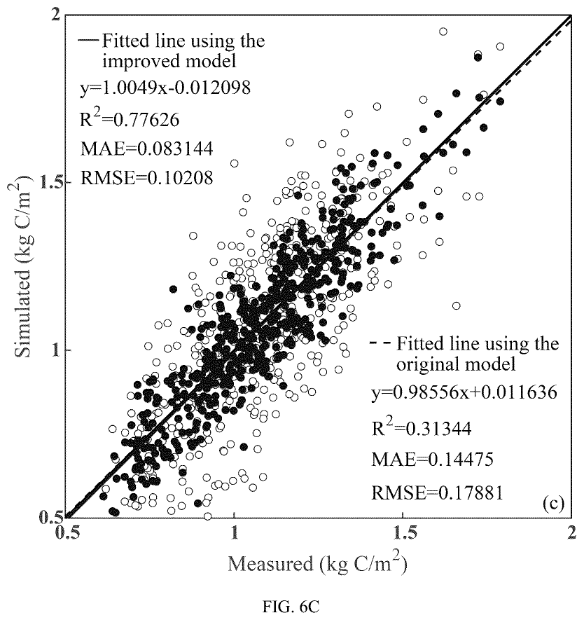

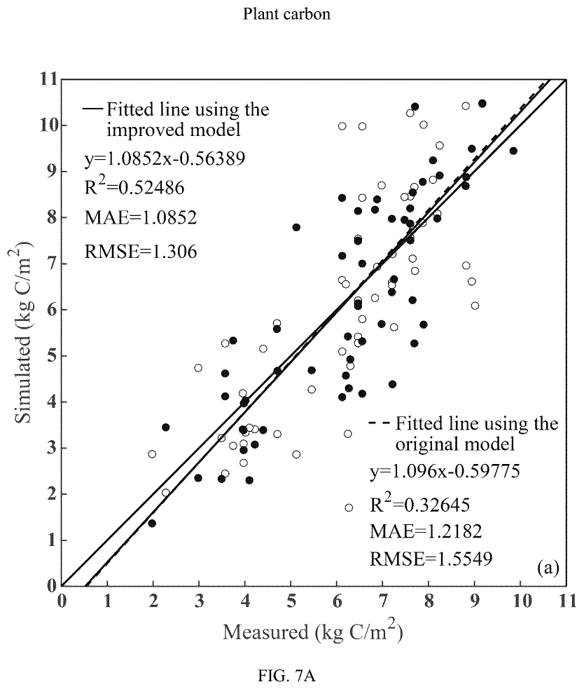

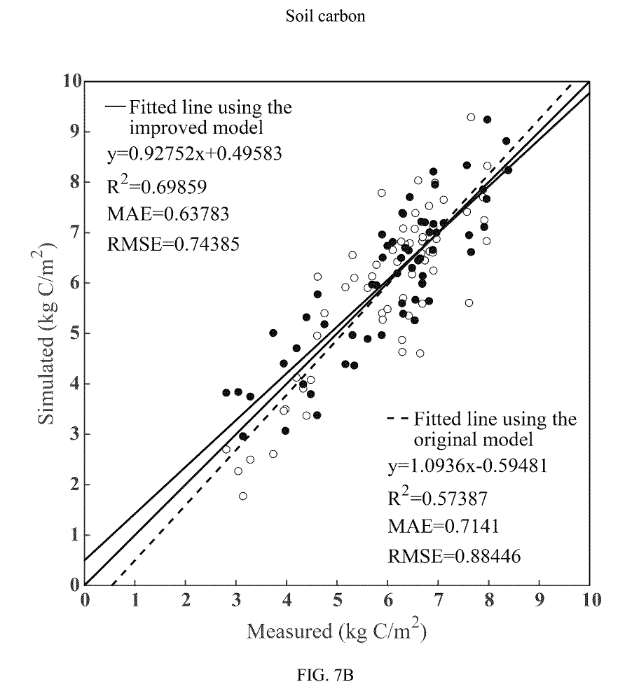

As shown in FIGS. 6 and 7, in a first step, the phenology module of the original model is set as evergreen phenology and deciduous phenology respectively for simulation, weighted average is performed according to the ratio of the mixed forest, and a simulation result is compared with a simulation result of an improved model formed by adjusting the phenology module. In a second step, the simulation result of the improved model formed by adjusting the phenology module is compared with a simulation result of an improved model formed by adjusting the phenology module and adding the thinning module. All simulation processes are performed based on optimized eco-physiological parameters.

In the present disclosure, R², MAE, and RMSE are calculated to estimate the simulation result:

$${R}^{2} = {[ \frac{{\sum }_{i = 1}^{n} ( {simulated}_{i} - {simulated}_{min} ) ( {observed}_{i} - {observed}_{min} )}{\sqrt{{\sum }_{i = 1}^{n} {( {simulated}_{i} - {simulated}_{min} )}^{2} {( {observed}_{i} - {observed}_{min} )}^{2}}} ]}^{2}$$

$$MAE = \frac{1}{n} {\sum }_{i = 1}^{n} \vert {simulated}_{i} - {observed}_{i} \vert$$

$$RMSE = \sqrt{\frac{1}{n} {\sum }_{i = 0}^{n} {( {simulated}_{i} - {observed}_{i} )}^{2}}$$

where, n denotes a number of measured data; R² reflects a degree of fitting; MAE and RMSE can reflect a degree of difference; R² closer to 1 leads to lower MAE and RMSE, indicating a higher simulation accuracy.
S5. Sensitivity of the eco-physiological parameter is analyzed by an extended Fourier amplitude sensitivity test (EFAST) method.

The present disclosure analyzes the sensitivity of the eco-physiological parameters using the extended Fourier amplitude sensitivity test (EFAST) method. EFAST is a variance-based global sensitivity analysis algorithm that combines the computational efficiency of the features from accelerated segment test (FAST) method with the overall sensitivity of Sobol' method (Sobol, 1993) to quantify the sensitivity of each parameter and their interaction to the result. EFAST is widely used in sensitivity analysis of nonlinear models, such as hydrological models, crop growth models, and Biome-BGC. The sensitivity is acquired by estimating the variance contribution rate of each input parameter (Xᵢ) with a corresponding value range on the simulation result (Y):

$$Y = f ( X ) = f ( {X}_{1} , {X}_{2} , {X}_{3} , … , {X}_{n} )$$

where, Y denotes an output result (plant carbon storage, soil carbon storage, litterfall carbon storage, and total carbon storage) of the improved Biome-BGC model; and Xᵢ denotes each eco-physiological parameter within a given distribution range.

A total variance of a model output is expressed as follows:

$${V}_{Y} = {\sum }_{i} {V}_{i} + {\sum }_{i} {\sum }_{j > i} {V}_{ij} + {\sum }_{i} {\sum }_{j > i} {\sum }_{k > j} {V}_{ijk} + … + {V}_{12 … n}$$

where, V_{Y} denotes the total variance of the output; Vᵢ denotes a variance of a single parameter; and Vᵢⱼ−V_{12 . . . n} denotes a variance of an interaction between parameters.

The sensitivity is measured by the contribution of a given input factor to the variance of the output result. The present disclosure selects the first-order sensitivity index (Sᵢ) and the global sensitivity index (S_{iT}) to quantify the contribution of the input parameter to the output result. The first-order sensitivity index represents the direct contribution of the parameter to the total variance of the output result, and is calculated as follows:

$${S}_{i} = {V}_{i} / {V}_{Y}$$

The global sensitivity index is a sum of the first-order sensitivity of the parameter and sensitivity indices of each order for the interaction between the parameter and another parameter, and it is calculated as follows:

$${S}_{i T} = {S}_{i} + {S}_{ij} + {S}_{ijk} + … + {S}_{12 … n}$$

In the present disclosure, first, 37 parameters are selected for sensitivity analysis based on the actual situation (Table 1). According to the distribution range of each input parameter, each parameter is randomly sampled using Monte Carlo method, with a sampling frequency of 130 times for each parameter. Therefore, the sampling frequency for the eco-physiological parameters is 4,810 (130×37) times. Based on the generated multiple sets of input parameters, Biome-BGC is run in batches to simulate the carbon storage of the pine-oak mixed forests from 1980 to 2019, and calculate the average carbon storage over 40 years as the final model input. The sensitivity and uncertainty analysis software SimLab2.2 is used to analyze the sensitivity of the eco-physiological parameters in Biome-BGC. Finally, the sensitivity indicators are divided into three levels, including highly sensitive parameters greater than 0.2, moderately sensitive parameters of 0.1 to 0.2, and insensitive parameters less than 0.1.

S6. A highly sensitive parameter is selected, and statistical analysis is performed by a path analysis method to acquire a result.

After the highly sensitive parameters are selected, the path analysis method is used to explore the influence of parameters and their interactions on the output. Path analysis is a multivariate statistical analysis method that can reflect the causal relationships between variables in the model. The path coefficient can reflect the strength of the causal relationships between variables. In the present disclosure, the path analysis is performed using the lavaan package in R language.

The optimization results of the 17 eco-physiological parameters of the pine-oak mixed forest are shown in Table 4.

TABLE 4
Optimal values of the 17 eco-physiological
parameters of the pine-oak mixed forest

| Parameter | Unit | Range | Optimal value |
|---|---|---|---|
| Tₜ_{_{—}}_{d} | prop. | [0, 1] | 0.5 |
| LFG_{d} | prop. | [0, 1] | 0.25847 |
| LFRT | 1/year | [0, 1] | 0.0018768 |
| WPM | 1/year | [0.0, 0.1] | 0.016064 |
| FRC:LC | ratio | [0.5, 1.5] | 0.5774 |
| SC:LC | ratio | [1, 4] | 3.2783 |
| CRC:SC | ratio | [0.184, 0.232] | 0.1953 |
| CGP | prop. | [0, 1] | 0.71716 |
| C: N_{leaf} | kgC/kgN | [18, 40] | 36.6798 |
| C: Nₗᵢₜₜₑᵣ | kgC/kgN | [41, 90] | 51.8905 |
| Wᵢₙₜ | 1/LAI/d | [0.0, 0.1] | 0.1 |
| LAl_{all:proj} | DIM | [1.5, 4.0] | 1.5 |
| SLA | m²/kgC | [10, 60] | 56.1132 |
| FLNR | DIM | [0, 1] | 1 |
| G_{cut} | m/s | [0.0, 0.0001] | 0 |
| LWPᵢ | MPa | [−0.78, −0.272] | −0.6 |
| VPDᵢ | Pa | [488, 1320] | 930 |

As shown in FIGS. 6A-6C, compared with the simulation of the original model, the improved model formed by modifying the phenology module significantly improves the accuracy of simulation in all three carbon components (vegetation carbon, litterfall carbon, and soil carbon). The R² values of the three carbon components increase by 0.33, 0.21, and 0.46, respectively, with vegetation carbon and soil carbon increasing by more than twice. Similarly, the reduction in MAE and RMSE is also significant (20% to 76%). In addition, the new model improves the slope of the 1:1 linear relationship between the measured data and simulated data of the three carbon pools. The simulation results of the improved model formed by modifying the phenology module are reasonable and thus this improved model can be applied to simulate the carbon storage of the pine-oak mixed forest.

As shown in FIGS. 7A-7B, due to the lack of validation data for litterfall carbon storage, the present disclosure uses the validations of plant carbon storage and soil carbon storage to evaluate the simulation accuracy of the improved model formed by modifying the phenology module as well as the improved model formed by modifying the phenology module and introducing the thinning module. In the improved model formed by modifying the phenology module, the R², MAE, and RMSE between the simulated and measured values of the plant carbon storage are 0.33, 1.21, and 1.55 kg C/m², respectively, while those of the soil carbon storage are 0.57, 0.71, and 0.88 kg C/m², respectively. In the improved model formed by modifying the phenology module and introducing the thinning module, the R², MAE, and RMSE between the simulated and measured values of the plant carbon storage are 0.52, 1.09, and 1.31 kg C/m², respectively, while those of the soil carbon storage are 0.70, 0.64, and 0.74 kg C/m², respectively. The improved model formed by modifying the phenology module and introducing the thinning module outperforms the improved model formed by only modifying the phenology module in simulating the plant carbon storage and soil carbon storage.

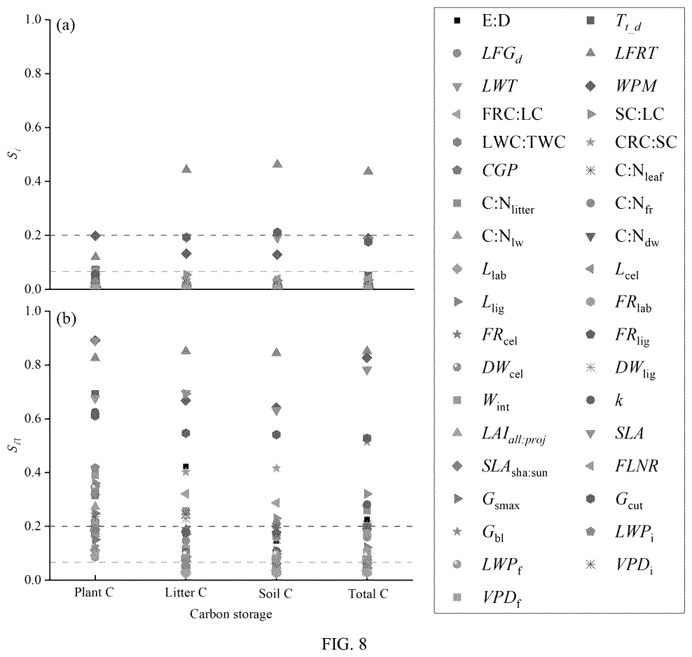

The sensitivity analysis of the carbon storage is shown in FIG. 8, where the highly sensitive parameters are located above the black dashed line, and the moderately sensitive parameters are displayed above the gray dashed line and below the black dashed line. LFRT has the highest Sᵢ and S_{iT} in the litterfall carbon storage, the soil carbon storage, and the total carbon storage, and the carbon storage can be controlled by directly or interactively. There is no highly sensitive parameter for the plant carbon storage in Sᵢ, and it is most affected by WPM. In S_{iT}, WPM, LFRT, WPM, SC:LC, CRC:SC, C:Nₗᵢₜₜₑᵣ, SLA, and G_{cut} exhibit significant control over the four carbon pools. In the four carbon pools, there are more sensitive parameters in S_{iT} than in Sᵢ, indicating that many parameters affect the carbon storage only through interactions, especially in the plant carbon storage.

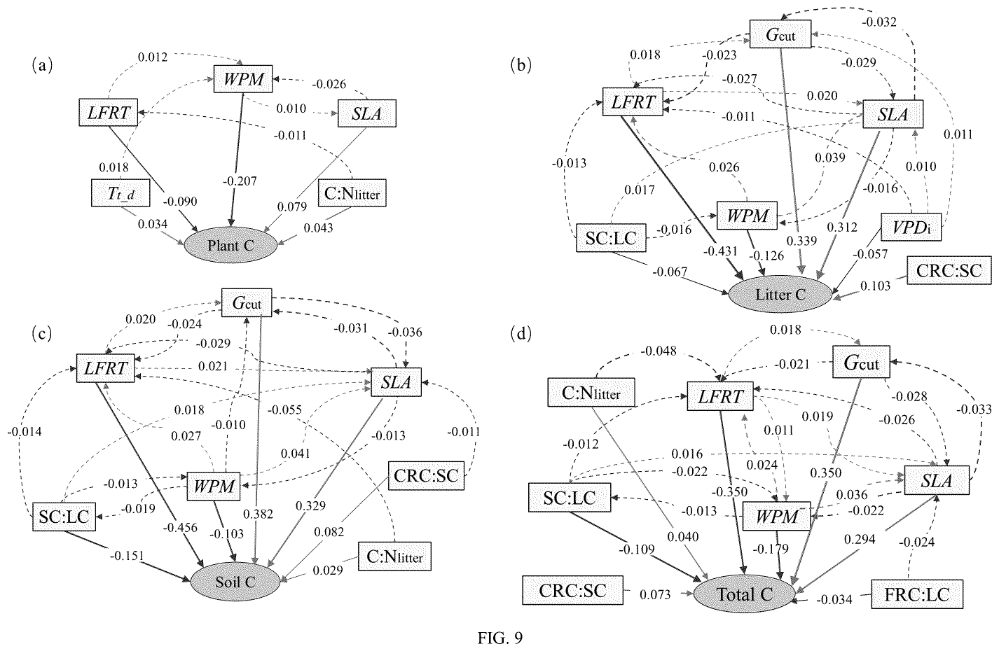

FIG. 9 shows the impact of the eco-physiological parameters on the carbon storage. For the litterfall carbon storage, the soil carbon storage, and the total carbon storage, G_{cut} and SLA have a direct positive effect, with G_{cut} having the greatest impact. WPM generates a significant indirect positive effect through SLA. LFRT, WPM, and SC: LC all have negative direct effects, with LFRT showing the highest performance. G_{cut} and SLA have a greater indirect negative effect through each other. C:Nₗᵢₜₜₑᵣ has a significant indirect negative effect on the soil carbon storage and total carbon storage through LFRT. For the plant carbon storage, C:Nₗᵢₜₜₑᵣ, SLA, and T_{t_d} show a direct positive effect, while LFRT and WPM show a direct negative effect. SLA has a significant indirect negative effect through WPM.

In FIG. 9, all path coefficients are standardized. In the figure, the solid line represents the direct impact, while the dashed line represents the indirect impact; the green line represents the positive effect, while the red line represents the negative effect; and the width of the arrow is directly proportional to the magnitude of the standardized path coefficient (P<0.01).

What is claimed is:

1. A method for calculating a carbon storage in a mixed forest ecosystem, comprising the following steps:
S1: acquiring basic geographic data, meteorological data, an eco-physiological parameter, thinning management history data, and validation data;
S2: proposing an improved biome-biogeochemical cycles (Biome-BGC) model suitable for simulating the carbon storage of the mixed forest ecosystem under a management by improving a phenology module, adding a thinning operation management module, and optimizing the eco-physiological parameter, based on an existing Biome-BGC model, specifically comprising:
S201: developing a phenology model suitable for simulating a mixed forest by improving the phenology module based on an evergreen phenology model, specifically comprising:
S2011: calculating start and end times of a transfer period of deciduous vegetation by defining a first parameter, wherein the first parameter is a proportion of a transfer growth period to a growing season of the deciduous vegetation;
S2012: calculating a daily transfer amount of the mixed forest in different phenological periods by defining a second parameter, wherein the second parameter is a ratio of evergreen vegetation to the deciduous vegetation; and
S2013: describing start and end times of a litterfall process of the deciduous vegetation by defining a third parameter, wherein the third parameter is a proportion of the litterfall process to the growing season of the deciduous vegetation; and calculating a daily litterfall amount of the mixed forest in different phenological periods based on the ratio of the evergreen vegetation to the deciduous vegetation;
S202: adding the thinning operation management module to simulate an impact of thinning on the carbon storage of the mixed forest ecosystem; and
S203: optimizing and analyzing the eco-physiological parameter through a flower pollination algorithm (FPA), and establishing a set of parameters suitable for simulating the carbon storage of the mixed forest ecosystem;
S3: simulating, by taking the mixed forest as a research object, the carbon storage based on the improved Biome-BGC model;
S4: validating the improved Biome-BGC model;
S5: analyzing a sensitivity of the eco-physiological parameter by an extended Fourier amplitude sensitivity test (EFAST) method; and
S6: selecting a highly sensitive parameter, and analyzing positive and negative effects of the highly sensitive parameter on the carbon storage by a path analysis method.

2. The method for calculating the carbon storage in the mixed forest ecosystem according to claim 1, wherein in the step S1:
the basic geographic data comprises: a digital elevation model (DEM), a slope, an aspect, a soil sand content, a clay content, a silt content, a shortwave albedo, a nitrogen deposition, and a nitrogen fixation;
the meteorological data comprises: a daily maximum temperature, a daily minimum temperature, a daily average temperature, a daily precipitation, a daily saturated vapor pressure deficit, a daily shortwave radiation flux density, and a day length;
regarding the eco-physiological parameter, the first parameter, the second parameter, and the third parameter, comprising the ratio of the evergreen vegetation to the deciduous vegetation, the proportion of the transfer growth period to the growing season of the deciduous vegetation, and the proportion of the litterfall process to the growing season of the deciduous vegetation, are added based on the eco-physiological parameter defined by the existing Biome-BGC model to calculate the daily transfer amount and the daily litterfall amount;
regarding the thinning management history data, 10 parameters are defined in the thinning operation management module, comprising a thinning day, as well as thinning rates and transport rates of various plant organs; and
the validation data involves a validation based on measured data.

3. The method for calculating the carbon storage in the mixed forest ecosystem according to claim 1, wherein in the step S2011: calculating the start and end times of the transfer period of the deciduous vegetation by defining the first parameter, wherein the first parameter is the proportion of the transfer growth period to the growing season of the deciduous vegetation:
for the deciduous vegetation, the transfer period is described based on a start and an end of the growing season; when a sum of daily average soil temperatures exceeds a defined critical value, a leaf starts to expand; and an actual leaf onset day is 15 days earlier than a calculated leaf onset day, marking the start of the growing season:
$$S {T}_{soil} = {\sum }_{i = 1}^{m} {Tsoil\_avg}_{i} ( when Tsoil\_avg > 0 , m \leq 365 )$$
$${T}_{crit} = {e}^{4.795 + 0.129 * {T}_{avg}}$$
$${onset}_{day} = m ( {ST}_{soil} \geq {T}_{crit} )$$
$${Act}_{onset \_ day} = {onset}_{day} - 15$$
wherein Tsoil_avgᵢ denotes an average soil temperature on an i-th day of a year; STₛₒᵢₗ denotes a sum of daily average soil temperatures when an average soil temperature is greater than 0; T_{avg} denotes an average of daily average temperatures within operating days; T_{crit} denotes the defined critical value; onset_{day} denotes the calculated leaf onset day; Act_{onset_day} denotes the actual leaf onset day; and m denotes a day when STₛₒᵢₗ is greater than or equal to T_{crit};
if, after July 1st, a day length is less than 10 hours and 55 minutes, and a soil temperature is lower than an average soil temperature in autumn or lower than 2° C., all leaves fall; and an actual leaf offset day is 15 days later than a calculated leaf offset day, marking the end of the growing season;
all the leaves fall when one of following conditions is met:
$$\{ \begin{matrix}\begin{matrix}{Daylen}_{j} \leq 39300 AND \\ {Tsoil\_avg}_{j} \leq {Tsoil}_{avg \_ aut} ( Sept . and Oct . ) ( j \geq 182 )\end{matrix} \\ {Tsoil\_avg}_{j} < 2 ( j \geq 182 )\end{matrix}$$
$${offset}_{day} = j$$
$${Act}_{offset \_ day} = {offset}_{day} + 15$$
wherein Daylenⱼ denotes a day length of a j-th day of the year; Tsoil_avgⱼ denotes an average soil temperature on the j-th day of the year; Tsoil_{avg_aut} denotes an average soil temperature between September and October; offset_{day} denotes the calculated leaf offset day; Act_{offset_day} denotes the actual leaf offset day; and the growing season is calculated based on the actual leaf onset day and the actual leaf offset day;
$$ngrowthdays = {Act}_{offset \_ day} - {Act}_{onset \_ day}$$
$${t}_{1} = {Act}_{onset \_ day}$$
$${t}_{2} = {Act}_{offset \_ day} + \times {T}_{f \_ d}$$
wherein ngrowthdays denotes a number of days in the growing season; t₁ and t₂ denote a start day and an end day of the transfer period of the deciduous vegetation, respectively; and T_{t_d} denotes the proportion of the transfer growth period to the growing season of the deciduous vegetation.

4. The method for calculating the carbon storage in the mixed forest ecosystem according to claim 1, wherein in the step S2012: calculating the daily transfer amount of the mixed forest in different phenological periods by defining the second parameter, wherein the second parameter is the ratio of the evergreen vegetation to the deciduous vegetation:
$${S}_{daily \_ transfer} = \{ \begin{matrix}{C}_{transfer} / ndays\_E ( 1 \leq nday \leq {t}_{1} ) \\ \begin{matrix}\frac{E : D}{1 + E : D} \times {C}_{transfer} / ndays\_E + \frac{1}{1 + E : D} \times \\ 2 \times {C}_{transfer} / ndays\_D ( {t}_{1} \leq nday \leq {t}_{2} )\end{matrix} \\ {C}_{transfer} / ndays\_E ( {Act}_{offset \_ day} \leq nday \leq 365 )\end{matrix}$$
wherein S_{daily_transfer} denotes the daily transfer amount of the mixed forest; C_{transfer} denotes a transfer amount of each plant organ in the mixed forest; ndays_E denotes a number of remaining days for a transfer of the evergreen vegetation; ndays_D denotes a number of remaining days for a transfer of the deciduous vegetation; E:D denotes the ratio of the evergreen vegetation to the deciduous vegetation; and nday denotes a day of a year.

5. The method for calculating the carbon storage in the mixed forest ecosystem according to claim 1, wherein in the step S2013: describing the start and end times of the litterfall process of the deciduous vegetation by defining the third parameter, wherein the third parameter is the proportion of the litterfall process to the growing season of the deciduous vegetation:
$${t}_{3} = {Act}_{offset \_ day} - ( ngrowthdays \times {LFG}_{d} ) + 1$$
$${t}_{4} = {Act}_{offset \_ day}$$
wherein t₃ and t₄ denote a start day and an end day of the litterfall process of the deciduous vegetation, respectively; and LFG_{d} denotes the proportion of the litterfall process to the growing season of the deciduous vegetation;
wherein in the step: calculating the daily litterfall amount of the mixed forest in different phenological periods based on the ratio of the evergreen vegetation to the deciduous vegetation:
$${S}_{daily \_ litterfall} = \{ \begin{matrix}{C}_{litterfall \_ increment \_ E} ( 1 \leq nday \leq {Act}_{onset \_ day} ) \\ \frac{E : D}{1 + E : D} \times {C}_{litterfall \_ increment \_ E} ( {Act}_{onset \_ day} \leq nday \leq {t}_{3} ) \\ \begin{matrix}{C}_{litterfall \_ increment \_ E} \times \frac{E : D}{1 + E : D} \times \\ {C}_{litterfall \_ increment \_ D} \times \frac{1}{1 + E : D} ( {t}_{3} \leq nday \leq {t}_{4} )\end{matrix} \\ {C}_{litterfall \_ increment \_ E} ( {t}_{4} \leq nday \leq 365 )\end{matrix}$$
$${C}_{litterfall \_ increment \_ E} = \frac{E : D}{1 + E : D} \times {C}_{annmax} \times F\_turnover / 365.$$
$$Drate = 2. \times ( {C}_{leaf \_ froot} - {C}_{litterfall \_ increment {\_ D}_{t}} \times {litdays}_{D}^{2}$$
$${C}_{litterfall \_ increment {\_ D}_{t + 1}} = {C}_{litterfall \_ increment {\_ D}_{t}} + Drate$$
wherein S_{daily_litterfall} denotes the daily litterfall amount; C_{litterfall_increment_E} denotes a daily litterfall amount from the evergreen vegetation, remaining constant throughout the year; C_{litterfall_increment_D} denotes a daily litterfall amount from the deciduous vegetation; Cₐₙₙₘₐₓ denotes an annual maximum daily carbon content; F_turnover denotes an annual turnover rate; C_{leaf_froot} denotes a carbon content in a leaf or fine root; litdays_{D} denotes a number of remaining days for the deciduous vegetation to fall; Drate denotes a linear growth rate; t denotes a number of days required to remove all fine roots and leaves, as well as a number of iterations in an equation; C_{litterfall_increment_D}_{ₜ} denotes a daily litterfall amount from the deciduous vegetation on a t-th day; and C_{litterfall_increment_D}_{ₜ₊₁} denotes a daily litterfall amount from the deciduous vegetation on a (t+1)-th day.

6. The method for calculating the carbon storage in the mixed forest ecosystem according to claim 1, wherein the step S202: adding the thinning operation management module to simulate the impact of the thinning on the carbon storage of the mixed forest ecosystem comprises:
defining a thinning day, as well as thinning rates and transport rates of various plant organs, wherein the thinning rate refers to a proportion of biomass removed during the thinning, while the transport rate refers to a proportion of biomass removed from a site after thinning and no longer participating in carbon cycling after transportation is completed; and
calculating, based on the thinning rate and the transport rate of each plant organ, a carbon loss and a carbon content entering a next litterfall pool, wherein:
$${C}_{all \_ to \_ THN} = {C}_{all} \times THN$$
$${C}_{all \_ to \_ THN \_ litr} = {C}_{all \_ to \_ THN} \times ( 100 - TRAN ) / 100 \times litter$$
wherein THN denotes the thinning rate; Cₐₗₗ denotes a carbon content of each plant organ; C_{all_to_THN} describes a carbon flux generated by thinning of each plant organ and is considered as a carbon loss of each plant organ; TRAN denotes the transport rate of each plant organ; litter denotes a ratio of each plant organ entering four litterfall pools; and C_{all_to_THN_litr} describes carbon remaining on site after thinning, and the carbon is converted into a corresponding litterfall component.

7. The method for calculating the carbon storage in the mixed forest ecosystem according to claim 1, wherein the step S203: optimizing and analyzing the eco-physiological parameter through the FPA, and establishing the set of parameters suitable for simulating the carbon storage of the mixed forest ecosystem comprises:
setting a maximum number of iterations, a population size, and a transfer probability; setting an objective function as a minimum residual sum between a simulated value and a measured value of the carbon storage, with a decision variable being the eco-physiological parameter to be optimized; and repeatedly adjusting a value of the eco-physiological parameter within a feasible range until the objective function reaches an ideal minimum value.

8. The method for calculating the carbon storage in the mixed forest ecosystem according to claim 1, wherein the step S3: simulating, by taking the mixed forest as the research object, the carbon storage based on the improved Biome-BGC model comprises:
first step: performing a spin-up process to make the improved Biome-BGC model enter a stable state and output a restart file; and second step: inputting the restart file into the improved Biome-BGC model, running the improved Biome-BGC model forward from hence, and starting the thinning operation management module in an area, wherein a thinning operation management is implemented in the area to simulate the impact of the thinning on the carbon storage.

9. The method for calculating the carbon storage in the mixed forest ecosystem according to claim 1, wherein the step S4: validating the improved Biome-BGC model comprises:
first step: setting the phenology module of the existing Biome-BGC model as evergreen phenology and deciduous phenology respectively for simulation, performing weighted average according to a ratio of the mixed forest to obtain a first simulation result, and comparing the first simulation result with a second simulation result of the improved Biome-BGC model formed by adjusting the phenology module; and
second step: comparing the second simulation result of the improved Biome-BGC model formed by adjusting the phenology module with a third simulation result of the improved Biome-BGC model formed by adjusting the phenology module and adding the thinning operation management module, wherein all simulation processes are performed based on an optimized eco-physiological parameter; and
calculating R², mean absolute error (MAE), and root mean square error (RMSE) to evaluate the second simulation result or the third simulation result of the improved Biome-BGC model:
$${R}^{2} = {[ \frac{{\sum }_{i = 1}^{n} ( {simulated}_{i} - {simulated}_{min} ) ( {observed}_{i} - {observed}_{min} )}{\sqrt{{\sum }_{i = 1}^{n} {( {simulated}_{i} - {simulated}_{min} )}^{2} {( {observed}_{i} - {observed}_{min} )}^{2}}} ]}^{2}$$
$$MAE = \frac{1}{n} {\sum }_{i = 1}^{n} \vert {simulated}_{i} - {observed}_{i} \vert$$
$$RMSE = \sqrt{\frac{1}{n} {\sum }_{i = 0}^{n} {( {simulated}_{i} - {observed}_{i} )}^{2}}$$
wherein n denotes a number of measured data; R² reflects a degree of fitting; MAE and RMSE reflect a degree of difference; R² closer to 1 leads to lower MAE and RMSE, indicating a higher simulation accuracy.

10. The method for calculating the carbon storage in the mixed forest ecosystem according to claim 1, wherein in the step S5: analyzing the sensitivity of the eco-physiological parameter by the EFAST method:
$$Y = f ( X ) = f ( {X}_{1} , {X}_{2} , {X}_{3} , … , {X}_{n} )$$
$${V}_{Y} = {\sum }_{i} {V}_{i} + {\sum }_{i} {\sum }_{j > i} {V}_{ij} + {\sum }_{i} {\sum }_{j > i} {\sum }_{k > j} {V}_{ijk} + … + {V}_{12 … n}$$
$${S}_{i T} = {S}_{i} + {S}_{ij} + {S}_{ijk} + … + {S}_{12 … n}$$
wherein Y denotes an output result of the improved Biome-BGC model; Xᵢ denotes each eco-physiological parameter within a given distribution range; V_{Y} denotes a total variance of the output result; Vᵢ denotes a variance of a single eco-physiological parameter; Vᵢⱼ−V_{12 . . . n} denotes a variance of an interaction between the eco-physiological parameters; Sᵢ denotes a first-order sensitivity index, indicating a direct contribution of the eco-physiological parameter to the total variance of the output result; and S_{iT} denotes a global sensitivity index, representing a sum of the first-order sensitivity index of the eco-physiological parameter and sensitivity indices of each order for the interaction between the eco-physiological parameters.
